(19) United States
(12) Patent Application Publication
(10) Pub. No.: US 2006/0094643 A1
(43) Pub. Date: May 4, 2006

(54) Compositions of hyaluronic acid and methods of use

(76) Inventors: Yuri Svirkin, Winchester, MA (US); Ramine Parsa, Waltham, MA (US); Dmitry Zingerman, North Quincy, MA (US)

Correspondence Address: WOLF GREENFIELD & SACKS, PC;FEDERAL RESERVE PLAZA, 600 ATLANTIC AVENUE, BOSTON, MA, 02210-2206, US

(21) Appl. No.: 10/519,329

(22) PCT Filed: Jul. 3, 2003
(86) PCT No.: PCT/US03/21034

§ 371 Date: Sep. 22, 2005

Related U.S. Application Data
(60) Provisional application No. 60/393,954, filed on Jul. 3, 2002.

(51) Int. Cl. A61K 38/16

(52) U.S. Cl. 514/8

(57) ABSTRACT

The invention provides compositions for the treatment of disorders characterized by dryness including dry eye and dry mouth. The compositions commonly comprise a conjugate of hyaluronic acid and polylysine. These conjugates are attached to affected body tissues or surfaces using transglutaminase, and preferably endogenous transglutaminase.

### FIELD OF THE INVENTION

The invention relates to compositions and methods for alleviating symptoms associated with disorders that benefit from the administration of hyaluronic acid, including but not limited to dry eye and dry mouth.

### BACKGROUND OF THE INVENTION

Dry eye is a condition of persistent dryness of the eye, including the cornea and conjunctiva. It can result from abnormal or inadequate tear formation, and deficiency in mucin secretion (i.e., keratoconjunctivitis sicca). Dry eye symptoms can be manifest as a result of various underlying disorders such as autoimmune disorders that damage lacrimal (i.e., tear-producing) glands, such as rheumatoid arthritis, Sjögren's syndrome, systemic lupus erythrematosus, and systemic sclerosis and sarcoidosis. Dry eye can also be induced following eye surgery, such as Lasik™ surgery. Dry eye is estimated to affect more than 13 million individuals in the United States.

Regardless of the underlying pathology, dry eye commonly involves the rapid breakdown of the pre-ocular tear film, resulting in dehydration of the exposed outer surface. Normal tear formation is required to keep the cornea and conjunctiva moist, and this in turn helps to prevent ulceration of both, as well as to maintain corneal transparency. In addition, tears facilitate movement of the eyelid over the eye surface (e.g., in blinking) and removal of foreign substances from the eye. Tears also normally contain lysozyme which is useful in preventing infection in the eye.

Dry eye can be associated with mild discomfort to severe pain in the eye. When it occurs for prolonged periods of time, it can cause blurred vision, grittiness and/or burning sensation, and itchiness. If the condition is allowed to persist without treatment, it can further lead to corneal ulcers and/or scarring.

To date, the most common form of treatment for dry eye is the use of artificial tears. Commercially available artificial tear products include Bion Tears (Alcon), Lacriset (Merck), Tears Naturale (Alcon) and Tears Naturale II (Alcon). One drawback to the use of artificial tears, however, is the need for frequent application, especially since the relief provided by artificial tear formulations is generally not long lasting.

Dry mouth, also known as xerostomia, is a condition characterized by inadequate production of saliva It can be a temporary condition caused by stress (e.g., fear), infection of the salivary (i.e., saliva-producing) gland, or the use of certain drugs such as anticholinergics, diuretics, antihistamines, clonidine, levodopa, methyldopa, and tricyclic antidepressants. It can also be a permanent condition of unknown etiology. Dry mouth has also been associated with Sjögren's syndrome and systemic sclerosis, and with radiation therapy of the mouth, neck and head (e.g., in the treatment of mouth cancer). Dry mouth generally also leads to difficulty and soreness in swallowing, speaking, and it can interfere with taste sensation. In some instances, it can also cause tooth decay.

Dry mouth is currently treated with mouth rinses, topical applications, salivary substitutes, or salivary stimulants such as sugarless candies. Saliva stimulants currently commercially available include cholinergic agonists such as Evoxac™ (cevimeline HCl, Daiichi Pharmaceutical Corp.) and Salagen® (pilocarpine HCl, MGI Pharma, Inc.). Commercially available saliva substitutes include Moi-Stir, Orex and Salivart. The most common treatment for alleviating dry mouth is spraying the inside of the mouth with artificial saliva. As with artificial tears, however, artificial saliva requires frequent application, and thus is cumbersome for affected individual.

Hyaluronic acid has been previously reported to be useful in the treatment of dry eye. However, such reports have focused on the use of free hyaluronic acid that, like the artificial tear treatments discussed above, must be continually applied.

### SUMMARY OF THE INVENTION

Prolonged activity treatments for dry eye and dry mouth, as well as other conditions that would benefit from hyaluronic acid administration, would be desirable as they would overcome the need for continual and frequent applications.

The present invention provides an alternative to the continual and frequent application of therapeutic agents for the treatment of dry eye, dry mouth, and other conditions associated with dryness. The invention is based, in part, on the discovery that the efficacy of hyaluronic acid in alleviating dry eye and dry mouth can be enhanced by covalently attaching hyaluronic acid to the affected body surface or tissue. Such attachment reduces the need for repeated and frequent application of dry eye or dry mouth agents because the hyaluronic acid is less likely to be rinsed away in the process of blinking or swallowing. According to the invention, hyaluronic acid is attached to the affected body surface via a linking molecule that is a substrate of transglutaminase. The linking molecule may include amino and/or carboxamide groups that are substrates of transglutaminase. The transglutaminase is preferably endogenous transglutaminase, but it may also be exogenous transglutaminase.

The compositions provided herein however are not solely limited to use in dry eye and dry mouth. Rather, they will find utility in other disorders characterized by dryness of other tissues including other mucosal tissues such as vagina, rectum, and nose (others to include), as well as external surfaces such as skin, hair, nail, and lip. In addition, the compositions find further utility in other body tissues such as endothelium (especially aortic endothelium), and bone joint cartilage. When used in vascular endothelium, the hyaluronic acid compositions provide long lasting prophylactic and/or therapeutic benefit given previous reports that hyaluronic acid inhibits platelet clotting. Hyaluronic acid has also been previously reported to reduce arthritic joint pain, and thus attachment of hyaluronic acid in bone joint cartilage would provide longer lasting relief from arthritis. When used in wrinkles caused by dryness of skin, hyaluronic acid would be applied topically, and would alleviate the need for frequent application of formulations of non-attachable hyaluronic acid. The invention intends to embrace formulations of the hyaluronic acid-linking molecule conjugate tailored to each of the foregoing conditions.

Thus, in one aspect, the invention provides a composition comprising a conjugate of hyaluronic acid and a linking molecule that is a substrate of transglutaminase, and free hyaluronic acid, wherein the free hyaluronic acid and the conjugate are present in a molar ratio of at least 2.

Various embodiments of the invention apply equally to the aspects disclosed herein. Accordingly, these embodiments will be recited once but it is to be understood that they apply to various aspects as taught in the present specification and claims.

In one embodiment, the linking molecule has at least two contiguous aliphatic amines, at least three contiguous aliphatic amines, at least four contiguous aliphatic amines, at least five aliphatic amines, or at least six aliphatic amines. In another embodiment, the linking molecule is native polylysine. In another embodiment, polylysine is selected from the group consisting of poly-L-lysine, poly-D-lysine, and poly-DL-lysine. In another embodiment, the linking molecule is a derivative of polylysine.

In one embodiment, the linking molecule has at least two continuous carboxamides, at least three contiguous carboxamides, at least four contiguous carboxamides, at least five carboxamides, or at least six carboxamides. In another embodiment, the linking molecule is native polyglutamine. In another embodiment, the linking molecule is selected from the group consisting of poly-L-glutamine, poly-D-glutamine, and poly-DL-glutamine. In still another embodiment, the linking molecule is a derivative of polyglutamine.

In one embodiment, the hyaluronic acid is native hyaluronic acid. In another embodiment, the hyaluronic acid is a derivative of hyaluronic acid selected from the group consisting of a pharmaceutically acceptable salt of hyaluronic acid, a hyaluronic acid ester, and a sulfated hyaluronic acid.

The molar ratio may be selected from the group consisting of at least 2.0 and at least 4.0.

In another embodiment, the composition is provided in a form selected from the group consisting of an eye dropper, a contact lens solution, an ophthalmic ointment, an eye pack, and a contact lens. In a further embodiment, the composition is provided in a form selected from the group consisting of a sublingual tablet, a mouthwash, a toothpaste, a candy, and an oral gel.

In one embodiment, the hyaluronic acid has a molecular weight of at least 100,000. In important embodiments, the conjugate has a negative charge to positive charge ratio of greater than 1.0.

In another embodiment, the composition further comprises a pharmaceutically acceptable carrier. In important embodiments, the pharmaceutically acceptable carrier has an osmolality of at least 280 mOsm. In other embodiments, the pharmaceutically acceptable carrier has a pH of at least 6.5.

The pharmaceutically acceptable carrier may comprise an ophthalmic preservative. In some embodiment, the ophthalmic preservative is selected from the group consisting of organic mercurials, quaternary ammonium compounds, parahydroxybenzoic acid esters, substituted alcohols and phenols. In a related embodiment, the organic mercurial is selected from the group consisting of phenylmercuric nitrate, phenylmercuric acetate, phenylmercuric borate, and thimerosal. In another related embodiment, the quaternary ammonium compound is selected from the group consisting of benzalkonium chloride, benzethonium chloride, cetyl pyridinium chloride, and polyquaternium-1 (POLYQUAD). In still another embodiment, the substituted alcohol and phenol is selected from the group consisting of chlorobutanol, and chlorobutanol/phenylethyl alcohol. The ophthalmic preservative may be an antibiotic.

In still another embodiment, the composition may further comprise an agent selected from the group consisting of a flavoring agent, a coloring agent and a scenting agent. In another embodiment, the composition further comprises arginine or fluoride.

In one embodiment, the conjugate has a weight ratio selected from the group consisting of at least 90%, at least 95%, and at least 99%. In another embodiment, the linking molecule is uncomplexed.

In another aspect, the invention provides a pharmaceutical composition comprising hyaluronic acid covalently linked to a linking molecule that is a substrate of transglutaminase, wherein the linking molecule is uncomplexed. In one embodiment, the composition comprises free hyaluronic acid. In another embodiment, the composition is provided in a form selected from the group consisting of an eye dropper, a contact lens solution, an ophthalmic ointment, an eye pack, and a contact lens. In still another embodiment, the composition is provided in a form selected from the group consisting of a sublingual tablet, a mouthwash, a toothpaste, a candy, and an oral gel.

In still another aspect, the invention provides a composition comprising a conjugate of hyaluronic acid and a to a linking molecule that is a substrate of transglutaminase, in an eye dropper bottle. In one embodiment, the composition further comprises a pharmaceutically acceptable carrier. In another embodiment, instructions for use are provided, optionally on the outside surface of the eye dropper bottle. In an important embodiment, the composition further comprises free hyaluronic acid. In other embodiments, the pharmaceutically acceptable carrier has an osmolality of at least 280 mOsm, and/or a pH of at least 6.5. In another embodiment, the pharmaceutically acceptable carrier comprises arginine or lysozyme. In an important embodiment, the linking molecule is uncomplexed.

In a further aspect, the invention provides a composition comprising a conjugate of hyaluronic acid and a linking molecule that is a substrate of transglutaminase, and an agent selected from the group consisting of a flavoring agent, a coloring agent and a scenting agent.

In one embodiment, the flavoring agent is selected from the group consisting of mannitol, sodium saccharin, magnasweet, peppermint extract, leaf power or oil; spearmint extract, leaf powder or oil; wintergreen oil; vanilla extract; parsley; oregano oil; bay leaf oil; clove oil; sage oil; sassafras oil; lemon oil; orange oil; anise oil; benzaldehyde; almond oil; camphor; cedar leaf oil; marjoram oil; cintronella oil; lavender oil; mustard oil; pine oil; pine needle oil; rosemary oil; thyme oil; cinnamon leaf oil; menthol; carvone; anethole; eugenol; methyl salicylate; limonene; cymene; n-decyl alcohol; citronellol; α-terpineol; methyl acetate; citronellyl acetate; methyl eugenol; cineole; linalool; eyktl linalool; vanillin; thymol; pellira oil; gaultheria oil; eucalyptus oil; caffeine, cream of tartar, lactic acid, malic acid, monosodium glutamate, nitrites, sorbitol, aspartame, acesulfame, dextrose, levulose, sodium cyclamate, stevioside, neo-hesperidyl dihydrochalcone, glycyrrhizin, perillartine, thaumatin, aspartylphenylalanine methyl ester, and p-methoxycinnamic aldehyde.

In another embodiment, the coloring agent is selected from the group consisting of FD&C Blue #1, FD&C Yellow #5, FD&C Yellow #10, FD&C Red #3, FD&C Red #40; caramel color or powder (#05439), chocolate shade (#05349), green lake blend (#09236), kowet titanium dioxide (#03970), yellow liquid color (#00403), and nitrites.

In still another embodiment, the scenting agent is selected from the group consisting of flower extract, herb extract, blossom extract, plant extract, and artificial scenting agents.

In important embodiments, the composition is formulated for oral or ocular administration. The composition may be formulated as a sublingual tablet, a mouth wash, a toothpaste, an oral gel, and a candy. The composition may further comprise arginine or fluoride.

The composition may further comprise a pharmaceutically acceptable carrier, optionally having a pH of at least 6.5, and/or an osmolality of greater than 280 mOsm.

In another aspect, the invention provides a composition comprising a conjugate of hyaluronic acid and a linking molecule that is a substrate of transglutaminase, in a carrier that comprises fluoride. In one embodiment, the carrier is a pharmaceutically acceptable carrier. In another embodiment, the pharmaceutically acceptable carrier has a pH of at least 6.5, and/or an osmolality of greater than 280 mOsm. In one embodiment, the conjugate is provided in a form selected from the group consisting of a sublingual tablet, a mouthwash, a toothpaste, a candy, and an oral gel.

In yet another aspect, the invention provides a composition comprising a conjugate of hyaluronic acid and a to a linking molecule that is a substrate of transglutaminase, in a sublingual tablet form. In a related embodiment, the sublingual form further comprises a sweetener selected from the group consisting of saccharin, aspartame, sorbitol, acesulfame, dextrose, levulose, sodium cyclamate, stevioside, neo-hesperidyl dihydrochalcone, glycyrrhizin, perillartine, thaumatin, aspartylphenylalanine methyl ester, and p-methoxycinnamic aldehyde. In another embodiment, the sublingual form comprises a vitamin or fluoride.

In still a further aspect, the invention provides a pharmaceutical composition comprising a conjugate of hyaluronic acid and a linking molecule that is a substrate of transglutaminase, and an effective amount of free hyaluronic acid, wherein the free hyaluronic acid and the conjugate are present in a molar ratio of at least 2.

The invention provides in other aspects, methods for treating or preventing an disorder characterized by dryness comprising administering an effective amount of any of the foregoing compositions to a subject in need thereof.

In one embodiment, the disorder is dry eye. In a related embodiment, the dry eye disorder is associated with a disorder selected from the group consisting of nonprogressive conjunctival cicatrization (Stevens-Johnson syndrome), Sjögren's syndrome, trachoma, and cicatricial pemphigoid. In another embodiment, the disorder is dry mouth. In yet another disorder, the subject has undergone or will undergo a surgical procedure that may induce dry eye symptoms such as corrective eye surgery (e.g., Lasik™ surgery).

In another aspect, the invention provides a method for treating a subject comprising administering to an eye of a subject having or at risk of having dryness of the eye an effective amount of a conjugate of hyaluronic acid and a linking molecule that is a substrate of transglutaminase. In one embodiment, the conjugate is provided in a form selected from the group consisting of an eye dropper, a contact lens solution, an ophthalmic ointment, an eye pack, and a contact lens.

In another aspect, the invention provides a method treating a subject comprising administering to an oral cavity of a subject having or at risk of having dryness of the oral cavity an effective amount of a conjugate of hyaluronic acid and a linking molecule that is a substrate of transglutaminase. In one embodiment, the conjugate is provided in a form selected from the group consisting of a sublingual tablet, a mouthwash, a toothpaste, a candy, and an oral gel.

In a further aspect, the invention provides a method of treating a subject comprising administering to a joint of a subject having or at risk of having joint discomfort an effective amount of a conjugate of hyaluronic acid and a linking molecule that is a substrate of transglutaminase.

In yet another aspect, the invention provides a method of treating a subject comprising administering to a blood vessel of a subject having or at risk of having excessive blood clotting or having an elevated risk of blood clotting an effective amount of a conjugate of hyaluronic acid and a linking molecule that is a substrate of transglutaminase.

In still a further aspect, the invention provides a method of treating a subject comprising administering to skin of a subject having or at risk of having wrinkles an effective amount of a conjugate of hyaluronic acid and a linking molecule that is a substrate of transglutaminase.

The foregoing compositions and conjugates are suitable in the methods provided herein.

The following embodiments apply equally to the foregoing methods. In one embodiment, the effective amount is less than 0.05 μg/kg per day.

These and other aspects and embodiments will be described herein in greater detail.

### BRIEF DESCRIPTION OF THE APPENDIX AND FIGURES

Appendix 1 is a table listing a variety of linkers that can be used in the conjugates of the invention.

FIG. 1A is a photograph of fluorescence microscopy of a rabbit cornea cross section 1 hour following the last administration of FITC-labeled polylysine in an eye drop formulation.

FIG. 1B is a photograph of fluorescence microscopy of a rabbit cornea cross section 36 hours following the last administration of FITC-labeled polylysine in an eye drop formulation.

FIG. 1C is a photograph of fluorescence microscopy of a rabbit cornea cross section 1 hour following the last administration of PCS-101 (FITC-labeled polylysine conjugated to hyaluronic acid, free hyaluronic acid, and buffer) in an eye drop formulation.

FIG. 1D is a photograph of fluorescence microscopy of a rabbit cornea cross section 36 hours following the last administration of PCS-101 (FITC-labeled polylysine conjugated to hyaluronic acid, free hyaluronic acid, and buffer) in an eye drop formulation.

FIG. 1E is a photograph of fluorescence microscopy of a rabbit cornea cross section 1 hour following the last administration of control vehicle alone in an eye drop formulation.

FIG. 1F is a photograph of bright field microscopy of a rabbit cornea cross section 36 hours following the last administration of control vehicle alone in an eye drop formulation.

FIG. 2 is a time course of conjugation to a human finger in vivo.

FIG. 3 is a compilation of photographs showing uptake of PCS-10-FITC after repeated applications to rabbit cornea in the absence of exogenously added transglutaminase.

FIG. 4 is a compilation of photographs showing uptake of PCS-10-FITC after repeated applications to rabbit cornea (cross section) in the absence of exogenously added transglutaminase.

FIG. 5 is a compilation of photographs of PCS-201 binding to pig palate.

FIG. 6 is a compilation of photographs of PCS-201 binding to the lower surface of pig tongue epithelium.

FIG. 7 is a compilation of photographs of PCS-201 binding to pig gum epithelium.

FIG. 8 is a compilation of photographs of PCS-201 binding to pig gum epithelium and pig tongue epithelium.

FIG. 9 is a compilation of photographs of PCS-201 binding to pig palate, pig gum epithelium and pig tongue epithelium.

FIG. 10 is a photograph of PCS-201 binding to pig mouth epithelium.

FIG. 11 is a compilation of photographs of crosslinking of PCS-201 and polylysine (both FITC labeled) to the inner lining of pig aortas.

FIG. 12 shows the effect of NaCl concentration on coupling of hyaluronic acid polylysine FITC to the cornified layer of rabbit cornea in the absence of exogenously added transglutaminase.

FIG. 13 shows the comparison of binding of PCS-101 and free HA to the superficial layer of rabbit cornea.

FIG. 14 is a bar graph comparing the corrected mean fluorescence intensity of HA-FITC conjugated to PLL-TRITC to HA-FITC and non-treated cells in a rabbit cornea model.

The figures are not required for enablement of the claimed invention.

### DETAILED DESCRIPTION OF THE INVENTION

The invention provides novel compositions and methods for the treatment of disorders that would benefit from the presence of hyaluronic acid, including disorders characterized by dryness. Disorders characterized by dryness include dry eye and dry mouth, which can result from insufficient tear and saliva production, respectively. In some embodiments, the invention uses hyaluronic acid as an active agent capable of moisturizing affected mucosal or external tissues, through its ability to attract and retain water molecules. As discussed in greater detail herein, hyaluronic acid reportedly has been used successfully in moisturizing the cornea and conjunctiva of subjects experiencing dry eye.

The invention is based, in part, on the discovery that hyaluronic acid can be attached to an affected surface such as a mucosal (e.g., cornea or oral cavity), endothelial, or external (e.g., hair or nail) surface using a transglutaminase-mediated reaction. Transglutaminases are a family of calcium-dependent enzymes mediating covalent cross-linking reactions between specific peptide bound γ-glutaminyl residues and various primary amino groups of peptide-bound lysines or polyamines, acting as amine donor substrates (Davies, et al., Adv. Exp. Med. Biol. 250, 391-401, 1988). In mammals, at least five enzymatically active transglutaminases have been identified, cloned and sequenced. The invention intends to embrace the use of any and all transglutaminases and enzymatic derivatives thereof that are capable of effecting covalent bonds between carboxamide groups of for example glutamine and amino groups of for example lysine. In preferred embodiments, the transglutaminase that effects the attachment of the conjugate to the body tissue is an endogenous transglutaminase, although in some embodiments exogenous transglutaminase may also be used.

Hyaluronic acid is not inherently a substrate of transglutaminase, as it contains no amino or carboxamide groups reactive with transglutaminase. It can be modified, however, according to the invention to render it susceptible to the action of transglutaminase. This may be accomplished, for example, by adding a carboxamide or amino side group(s) to an appropriate reactive group of hyaluronic acid (i.e., a “modified” hyaluronic acid). This can also be accomplished by covalently coupling glutamine, lysine or both glutamine and lysine to hyaluronic acid to form a conjugate that is a substrate of transglutaminase. The most preferred method is to couple a linking molecule, such as polyglutamine, polylysine, involucrin (a natural substrate of transglutaminase), or a fragment of involucrin, to hyaluronic acid to form an appropriate conjugate. Endogenous transglutaminases present in epithelial tissue such as the corneal epithelium or the oral epithelium are then able to catalyze covalent attachment of the linking molecule (with hyaluronic acid attached thereto) to amino or carboxamide substrates in the eye or oral cavity.

Native hyaluronic acid is a linear polymer of repeating monomers of disaccharides of D-glucuronic acid and N-acetyl D-glucosamine. As used herein, the term “hyaluronic acid” is intended to embrace native hyaluronic acid, as well as its derivatives (i.e., analogs) including but not limited to salts and esters, unless explicitly otherwise stated. Salts of hyaluronic acid include pharmaceutically acceptable salts such as sodium salts and ammonium quaternary salts. Hyaluronic acid derivatives include hyaluronic acid that has been modified by other chemical reactions such as esterification, and thus include hyaluronate esters, as well as sulfated hyaluronic acid (as described in U.S. Pat. No. 6,339,074 B1). An example of a hyaluronic acid derivative is hylan. Hyaluronic acid derivatives also include synthetic or semi-synthetic variants such as esters of hyaluronic acid and aliphatic, araliphatic, heterocyclic, and cycloaliphatic alcohols (e.g., benzyl or ethyl ester of hyaluronic acid) as described in U.S. Pat. Nos. 4,851,521; 4,965,353; and 5,202,431.

The compositions of the invention can further include hyaluronic acid in a free form in addition to the conjugated form. The use of free hyaluronic acid has previously been reported. It has now been discovered that hyaluronic acid can be attached to the eye or other affected surface via a transglutaminase-mediated linkage. Such attachment prolongs the time that hyaluronic acid is present at the eye (or other affected surface), and therefore reduces or eliminates completely the need to reapply hyaluronic acid to such surfaces. Thus, it is to be understood that the therapeutic benefit of the composition is mainly derived from the presence of the conjugated hyaluronic acid which once applied to a body tissue becomes covalently attached to that tissue. This is not the case for the free hyaluronic acid which may be present in the composition, since free hyaluronic acid has been demonstrated previously to have only limited therapeutic benefit.

As used herein, the term “conjugate” includes both direct and indirect attachment of the linking molecule to hyaluronic acid. Indirect attachment generally means that a spacer (i.e., a linker) exists between the linking molecule and the hyaluronic acid. Suitable spacers are described herein.

Although not intending to be bound by any particular theory or mechanism, hyaluronic acid is useful in treating disorders characterized by dryness, such as but not limited to dry eye, dry mouth and vaginal dryness, because it has the ability to act as a moisturizer and/or humectant. As used herein, a moisturizer is an agent that forms a film and can thereby trap water molecules and prevent or limit the extent of their evaporation. As used herein, a humectant is an agent that increases the water content of a tissue, surface or other region. Hyaluronic acid is generally negatively charged at neutral pH, and having hydrophilic hydroxy groups is able to attract and retain water molecules. Hyaluronic acid can both form a film and can attract and retain water molecules. Accordingly, it can function as both a moisturizer and humectant.

Hyaluronic acid has also been reported to inhibit platelet aggregation, and it is this mechanism which is exploited in embodiments of the invention relating to the vascular administration formulations.

Hyaluronic acid is commercially available, and sold under a variety of brand names including Healon, Hyalastine, Hyalectin, Hyloran (sodium hyaluronate), and Hyaloftil (high molecular weight hyaluronic acid). Alternatively, hyaluronic acid can be synthesized or purified from animal sources (such as rooster combs, commercially sold as Hyalform) or from in vitro fermentations (such as bacterial fermentation, commercially sold as Restylane), as described in U.S. Pat. Nos. 3,396,081; 3,862,003; 4,141,973; 4,517,296; 5,316,926; 6,090,596; among others.

The length of hyaluronic acid used is not critical to the invention, provided that it is of a length sufficient to hydrate the affected bodily surface or tissue. To that end, in some embodiments, shorter hyaluronic acid strands (i.e., having molecular weight less than 2000) will be less preferable than longer hyaluronic strands (i.e., having molecular weight greater than 100,000), unless there are several such shorter strands attached to the linking molecule. In instances in which the conjugate embraces only one linking molecule and one hyaluronic acid strand, longer hyaluronic acid strands are preferred at least in order to maximize the number of water molecules that can be retained. Methods for producing or isolating low and high molecular weight hyaluronic acid are known and have been reported in U.S. Pat. No. 4,141,973 (MW at least 750,000); U.S. Pat. No. 5,079,236 (MW 50,000 to 200,000); U.S. Pat. No. 5,316,926 (MW 1,100,000 to 4,000,000); U.S. Pat. No. 5,925,626 (MW between 50,000 and 100,000; and between 500,000 and 730,000); U.S. Pat. No. 6,090,596 (MW greater than 6,000,000); and U.S. Pat. No. 6,194,392 (MW between 150,000 and 750,000).

The length of hyaluronic acid strands will be referred to either as the number of disaccharide units (each unit having a molecular weight of approximately 401 Daltons), or the molecular weight of the strand. For example, a hyaluronic acid strand having a molecular weight of 200,000 is comprised of 498 disaccharide monomer units. In important embodiments, the hyaluronic acid strand has a molecular weight of greater than 50,000, greater than 60,000, greater than 75,000, greater than 100,000, greater than 150,000, and greater than 200,000. In still other embodiments, the hyaluronic acid can have higher a molecular weight of greater than 300,000, greater than 400,000, greater than 500,000, greater than 600,000, greater than 700,000, greater than 800,000, greater than 900,000, and greater than 1,000,000. In some preferred embodiments, the hyaluronic acid is at least 200,000, at least 210,000, at least 220,000, at least 230,000, at least 240,000, or at least 250,000.

In some embodiments, the conjugates of the invention are provided to a subject in combination with free hyaluronic acid. As used herein, “free” hyaluronic acid is not conjugated to a linking molecule. The invention does not rely on the binding of hyaluronic acid to its specific receptor (e.g., CD44) in order to achieve a therapeutic result. Rather, the localization of hyaluronic acid to affected sites is generally controlled through directed administration and the transglutaminase-mediated linkage. Binding of hyaluronic acid to its receptor is not required in the methods of the invention. The presence of free hyaluronic acid is not problematic as it does not reduce the efficacy of the conjugate because it does not compete with conjugated hyaluronic acid for binding to the cognate receptor. Moreover, the conjugates of the invention can be attached to any surface or tissue provided it contains sufficient levels of endogenous transglutaminase and transglutaminase substrates, regardless of whether it also expresses hyaluronic acid receptors.

In some embodiments, a hyaluronic acid derivative is used (either as the free form and/or the conjugated form) that binds to a hyaluronic acid receptor with affinity lower than that of native hyaluronic acid. In some embodiments, the affinity is less than 2-fold, less than 5-fold, less than 10-fold, less than 20-fold, less than 50-fold, or less than 100-fold the binding affinity of native hyaluronic acid.

In some embodiments, the molar ratio of free hyaluronic acid to conjugated hyaluronic acid in the composition is at least 10, at least 5, at least 4, at least 3.5, at least 3, at least 2.5, at least 2, at least 1.5, at least 1.2, at least 1, at least 0.9, at least 0.8, at least 0.7, at least 0.6, at least 0.5, at least 0.4, at least 0.3, at least 0.2, or at least 0.1. In some particular embodiments, the molar ratio of free hyaluronic acid to conjugated hyaluronic acid is greater than 1, preferably greater than 1.5, and even more preferably greater than 2. As used herein, the molar ratio of free hyaluronic acid to conjugated hyaluronic acid is the ratio of the number of moles of hyaluronic acid (including all MW variants) that is unconjugated to the linking molecule, to the number of moles of hyaluronic acid (of the same or different MW) that is conjugated to the linking molecule. Accordingly, in some embodiments, the amount of free hyaluronic acid is sufficient to compete with conjugated hyaluronic acid for binding to a hyaluronic acid receptor.

The linking molecules of the invention are substrates of transglutaminase. Accordingly, they possess aliphatic amines or carboxamides, but preferably not both. Preferred linking molecules are polymers bearing multiple reactive carboxamides and/or aliphatic amines that are substrates of transglutaminase. Carboxamide-containing compounds that are substrates of transglutaminase are well known and include glutamines. Aliphatic amines that are substrates of transglutaminase also are well known and are exemplified in, for example, U.S. Pat. No. 5,490,980, the disclosure of which is incorporated herein by reference. Unlike the '980 patent, however, which depicts single aliphatic amine moieties, the present invention involves in one aspect using a plurality of aliphatic amines. The aliphatic amines (or the plurality of aliphatic amines) may be contiguous, or they may be spaced apart at discrete intervals, preferably along the length of a branched or unbranched polymer. In some embodiments, the spacing of the reactive moieties is important for attaching the conjugates to a particular body tissue.

One embodiment involves linking molecules that are polymers having multiple units, each unit bearing an aliphatic amine that is a substrate of transglutaminase. The polymer can be a homopolymer or a heteropolymer. As used herein in connection with linking molecules, a polyaliphatic amine substrate of transglutaminase is a linking molecule with at least three aliphatic amines spaced apart from one another at discrete intervals along the backbone of the linking molecule, separated by one or more backbone atoms. This is most easily envisioned, for example, with polymers rich in lysine, whereby discrete units of the polymer carry the aliphatic amine, each being separately a substrate for transglutaminase. The linking molecule itself may be a polymer of contiguous lysines, preferably at least 2, at least 3, at least 4, and at least 5, or more, such contiguous lysines. Polymers of contiguous units, each carrying an aliphatic amine, are preferred. In some embodiments, the linking molecule may have as few as two contiguous lysine residues, and preferably, these are located at either end of the linking molecule.

Similarly, other important linking molecules are polymers having multiple units, each unit bearing a transglutaminase reactive carboxamide group. The polymer may be a homopolymer or a heteropolymer. A polycarboxamide substrate of transglutaminase is a linking molecule with at least two carboxamide spaced apart from one another at discrete intervals along the backbone of the linking molecule, separated by one of more backbone atoms. The linking molecule may be a polymer of contiguous carboxamides, preferably at least 2, at least 3, at least 4, or at least 5, or more, contiguous carboxamides. In some important embodiments, the contiguous carboxamides are located at either end of the linking molecule.

The most preferred linking molecules are polymers rich in a carboxamide moiety or an aliphatic amine moiety, such as glutamine or lysine, or glutamine and lysine. A polymer rich in carboxamide is one in which at least 20% of its units are carboxamide-carrying units. A polymer rich in aliphatic amines is one in which at least 20% of its units are aliphatic amine-carrying units. Accordingly, these polymers also embrace those having at least 30%, at least 40%, at least 50%, or more of its units so defined. A polymer rich in glutamine or lysine is a polymer with at least 20% of its units as glutamine or lysine, or glutamine and lysine. A polymer rich in carboxamides or aliphatic amines can also be a polymer that includes at least 3, preferably 4, and most preferably 5 or more separate and discretely spaced by a regular distance carboxamides or aliphatic amines, such as occurs with contiguous, linked glutamines or lysines. It should be understood, however, that a chain of as few as two glutamines or lysines can be attached to or tethered to hyaluronic acid to render it a substrate of transglutaminase. In preferred embodiments, the linking molecule and the conjugate that comprises it is a substrate for endogenous transglutaminase (i.e., it has sufficient transglutaminase reactive groups to be acted upon by endogenous transglutaminase).

One preferred linking molecule is polylysine. Polylysine includes poly-L-lysine, poly-D-lysine, and poly-DL-lysine. Another important linking molecule is poly-glutamine. Poly-glutamine includes poly-L-glutamine, poly-D-glutamine, and poly-DL-glutamine.

The linking molecules therefore include polylysine and polyglutamine in native or derivative form. A native form of polylysine is a polymer of lysine monomers. A derivative form of polylysine is a polymer of lysine monomers, one or more of which may be modified, either chemically or otherwise.

In some embodiments, the lining molecule is one that is not cleavable by proteases. These latter peptide derivatives can include backbone modifications that are not hydrolyzable.

The length of the linking molecule is generally not limiting, and both short and long linking molecules can be used in the invention. Accordingly, linking molecules as short as three residues (e.g., three lysines or three glutamines) can be used effectively. The molecular weight of the linking molecule can range from less than 200 Da to more than 100,000 Da, with the number of corresponding residues depending upon the make up of the linking molecule. Depending upon the embodiment, the linking molecule may have a molecular weight of at least 500, at least 1000, at least 5000, at least 10,000, at least 25,000, at least 50,000, at least 75,000, at least 100,000, or more. One of ordinary skill in the art will be able to ascertain the number of monomer units of the linking molecule based on its molecular weight and its composition. The linking molecule used in the Examples have an average length of 181 lysine residues, and thus an average molecular weight of about 23,000.

In important embodiments, the linking molecule is uncomplexed, particularly if it is polylysine. A linking molecule that is “uncomplexed” means that it is not associated with compounds other than the hyaluronic acid of the invention, and/or salts or amino acids of a vehicle solution. In important embodiments, an uncomplexed linking molecule is not complexed, for example, with a therapeutic agent such as a drug or a nucleic acid. The ability of polylysine to bind to nucleic acid to form an ionic complex has been reported. Since polylysine is positively charged at neutral pH, it tends to interact ionically with negatively charged agents such as nucleic acids. In some aspects of the invention, however, the conjugates are not intended to deliver nucleic acids and the linking molecules of such conjugates are uncomplexed.

Linking molecules such as polylysine can be maintained in an uncomplexed form, for example, by manipulating the salt concentration and pH in order to preclude the ionic interactions between polylysine and negatively charged agents (e.g., therapeutic agents such as drugs, or nucleic acids). For example, the concentration of anions can be increased in order to compete with the negatively charged agents for ionic binding to polylysine. In other embodiments, the conjugate can be provided in a slightly hypertonic solution, such as a osmolality that is greater than 280 mOsm. In other embodiments, the formulation has an osmolality of greater than 290 mOsm, greater than 300 mOsm, greater than 310 mOsm, greater than 320 mOsm, greater than 330 mOsm, greater than 340 mOsm, greater than 350 mOsm, or more. Any salt (including monovalent and divalent salts), amino acid or buffer can be used to adjust the osmolality of the solution.

The linking molecules can also be provided in an uncomplexed form by including other agents, such as but not limited to arginine. In some embodiments, these agents compete with lysine monomer units for binding to hyaluronic acid, and thereby preclude ionic complex formation between hyaluronic acid and polylysine components of the conjugate. Thus, in important embodiments, the hyaluronic acid and polylysine components are not ionically complexed to each other and rather are contacting each other solely at the point of conjugation (e.g., the covalent bond between them or that links both to a spacer molecule).

In still other embodiments, as the conjugates are not intended to carry nucleic acid molecules, nucleases such as DNases and RNases may be included in the formulations.

It is important that a sufficient number of transglutaminase reactive groups (i.e., aliphatic amines or carboxamides) are available for reaction to the tissue via the action of transglutaminase. Accordingly, not all transglutaminase reactive groups should be complexed, although in some instances, as few as 2-3 transglutaminase reactive groups may be sufficient for the transglutaminase-mediated reaction. In some embodiments, the transglutaminase reactive groups may be present at the end of the polymer, while in others they may be internally located.

The structure of the conjugate can vary in terms of both the hyaluronic acid and the linking molecule, provided that it has a sufficient number of amino or carboxamide reactive groups available (to render it a substrate of transglutaminase), and that the hyaluronic acid can moisturize the affected surface or tissue.

In its simplest form, the conjugate is a 1:1 conjugate of hyaluronic acid and linking molecule. That is, the conjugate would contain one strand of hyaluronic acid and one strand of linking molecule attached to each other either directly or indirectly.

In more complex forms, the conjugate contains several hyaluronic acid strands attached to a single linking molecule. The hyaluronic acids may be attached to the linking molecule at contiguous sites along the length of the linking molecule, or they may be attached at spaced intervals of consistent or random length. In this latter embodiment, the conjugate would be a graft copolymer having a linking molecule as its backbone and hyaluronic acid strands as its grafts. The length, number, and placement of hyaluronic acid side chains along the linking molecule backbone will affect the amount of conjugate that needs to be administered in order to impart therapeutic benefit. In some embodiments, a hyaluronic acid side chain can be grafted onto almost every available reactive group in the linking molecule, with the proviso that there must be sufficient transglutaminase reactive groups available in order to attach the conjugate to the affected body tissue or surface.

The transglutaminase reactive groups on the linking molecule that are used to attach the conjugate to the affected surface can be at the ends of the linking molecule but are not so limited. Accordingly, in one embodiment, the reactive groups can be in the middle of the linking molecule with hyaluronic acid strands attached thereto on one or both flanking sides.

The structure of the conjugate can also be described in terms of the extent of grafting of hyaluronic acid onto the linking molecule. As used herein, the grafting rate is expressed as the ratio of number of hyaluronic acid disaccharide monomers to number of linking molecule monomers. As an example, conjugation of one chain of hyaluronic acid having molecular weight of 220,000 (and containing about 549 hyaluronic disaccharide units) to a polylysine linking molecule having molecular weight of 23,000 (and containing about 181 lysine residues) corresponds to a grafting ratio of about 3 (i.e., 549/181). The grafting ratio can vary depending upon the number and length of hyaluronic acid chains grafted onto the linking molecule, and the length of the linking molecule itself. Depending upon the embodiment, therefore, the grafting ratio can range from below 0.001 and above 10000. In important embodiments, the grafting rate is at least 0.001, at least 0.005, at least 0.01, at least 0.1, at least 0.5, at least 1, at least 5, at least 10, at least 20, at least 30, at least 40, at least 50, at least 60, at least 70, at least 80, at least 90, at least 95, at least 100, at least 250, at least 500, at least 750, at least 1000, at least 2500, at least 5000, at least 7500, and at least 10000. It should be noted that grafting rate, per se, is not an indication of the number of linkages between the hyaluronic acid and the linking molecule. In some preferred embodiments, the number of hyaluronic disaccharide units is greater than the number of linking molecule subunits (e.g., lysine residues), and thus the ratio is greater than 1.

The conjugate can similarly be described structurally in terms of proportion of total weight of hyaluronic acid per total weight of the conjugate (i.e., the weight of the hyaluronic acid and the linking molecule), expressed as a percentage. This weight ratio can similarly range from less than 0.001 to 99.9%. In important embodiments, the weight ratio is at least 0.005%, at least 0.01%, at least 0.1%, at least 0.5%, at least 1%, at least 5%, at least 10%, at least 20%, at least 30, at least 40%, at least 50%, at least 60%, at least 70%, at least 80%, at least 90%, at least 95%, and at least 99%. In preferred embodiments in which the number of hyaluronic disaccharide monomers is in excess of the number of linking molecule monomers (e.g., lysine residues), the weight ratio will preferably be in excess of 90%. Thus, in preferred embodiments, the weight ratio is greater than 90%, greater than 91%, greater than 92%, greater than 93%, greater than 94%, greater than 95%, greater than 96%, greater than 97%, greater than 98%, greater than 99%, greater than 99.5%, and greater than 99.9%. Accordingly, the weight of linking molecule in a conjugate is generally 10% or less, in some important embodiments.

The molecular weight of the conjugate can also vary depending upon the composite of the conjugate. An exemplary conjugate having one chain of hyaluronic acid of molecular weight 220,000 and a linking molecule of molecular weight 23,000 has a molecular weight of 243,000. The molecular weight of the conjugate can vary from about 50,000 to in excess of 10,000,000, with a preferred range of 75,000 to 1,000,000, a more preferred range of 100,000 to 500,000, and an even more preferred range of 100,000 to 300,000.

In yet other instances, the conjugate can be described in terms of the proportion of hyaluronic acid (by weight) to polylysine (by weight), expressed as a percentage.

The conjugate can also be described with respect to its charge ratio (i.e., the negative to positive charge ratio). A charge ratio of less than 1 indicates an overall positive charge, while a charge ratio of greater than 1 indicates an overall negative charge. In important embodiments, the charge ratio can vary from greater than 1 to greater than 10. In some embodiments, the charge ratio can be greater than 1, greater than 2, greater than 3, greater than 4, greater than 5, greater than 6, greater than 7, greater than 8, greater than 9, greater than 10, greater than 12, or even greater. In other embodiments, the charge ratio ranges from 1 to 10, preferably from 2 to 8, more preferably from 3 to 7, and even more preferably from 4 to 6. In preferred embodiments, the conjugate has an overall negative charge, preferably at pH in the range of 6.5 to 8. At pH greater than 6.5, hyaluronic acid will be negative. At pH greater than about 8, lysine residues becomes neutrally charged, but the overall conjugate will still be negatively charged. For conjugates containing poly-glutamine, the conjugate will always be negatively charged over pH 6.5, because of the neutral charge of the glutamine residue.

As used herein, a conjugate means two entities stably bound to one another by any chemical or physiochemical means. It is important that the nature of the attachment be such that it does not impair substantially the effectiveness of hyaluronic acid or the substrate activity of the linking molecule. Keeping these parameters in mind, any linkage known to those of ordinary skill in the art may be employed including covalent or noncovalent. Covalent linkage is preferred. Such means and methods of attachment are well known to those of ordinary skill in the art.

In embodiments using polylysine as the linking molecule, the hyaluronic acid and polylysine are conjugated by a reductive amination reaction. The Examples provide the experimental protocol for forming polylysine and HA conjugates. Generally, the reductive end of hyaluronic acid is coupled with amino residues on polylysine to form a Schiff's base, followed by reduction to an imino bond. In one experimental design, hyaluronic acid and polylysine are dissolved in a solvent such as borate (pH 8.5) or phosphate (pH 8.3) buffer. A reductant such as sodium cyanoborohydride (NaBH₃CN) is added and the reaction is allowed to proceed for a time ranging from 1 hour to 5 days, at a temperature of 0 to 50° C. The reaction can be controlled by the addition of organic solvents such as dimethyl formamide or dimethyl sulfoxide. It can be enhanced by preventing ionic interaction between hyaluronic acid and polylysine. Such ionic interaction can be reduced by the addition of salts such as sodium chloride or potassium chloride, or by increasing the reaction temperature.

In some embodiments, the conjugate is formed using molar ratios of starting materials in the range of 0.5:1 to 5:1 of polylysine to hyaluronic acid. In one important embodiment, the starting molar ratio of polylysine to hyaluronic acid is 1.3:1. These reagent molar ratios are likely to give rise to conjugates having few (e.g., one or two) hyaluronic acid strands conjugated per polylysine.

Upon completion of the synthesis reaction, the solution may be dialyzed to remove unconjugated polylysine. However, the unconjugated hyaluronic acid preferably is not removed and thus the separation is one that selectively removes unconjugated polylysine but not unconjugated hyaluronic acid. For example, if the separation technique is dialysis, then the dialysis tubing is selected for a pore size that permits movement of the polylysine (having for example a MW of 23,000) but not hyaluronic acid (having for example a MW of at least 100,000).

In constructing conjugates, it may be desirable to tether the linking molecule to hyaluronic acid via a spacer. This can remove, for example, any problems that might arise from steric hindrance, wherein access by transglutaminase to the reactive moiety of the linking molecule is hindered. These spacers can be any of a variety of molecules, preferably nonactive, such as straight or even branched carbon chains of C₁-C₃₀, saturated or unsaturated, phospholipids, amino acids (e.g., glycine), and the like, naturally occurring or synthetic. Additional spacers include alkyl and alkenyl carbonates, carbamates, phosphates, and carbamides. These are all related and may add polar functionality to the spacers such as the C₁-C₃₀ previously mentioned. Suitable spacers such as those provided in Appendix A are commercially available for example from Pierce Chemical Co.

The conjugations or modifications described herein employ routine chemistry, which is well known to those skilled in the art of chemistry and thus does not form a part of the invention. The use of protecting groups and known linkers such as mono- and hetero-bifunctional linkers are well documented in the literature and will not be repeated here.

Attachment according to the invention thus need not be directed attachment. The components of the compositions of the invention may be provided with functionalized groups to facilitate their attachment and/or linker groups may be interposed between the components of these compositions to facilitate their attachment. In addition, the components of the compositions of the present invention may be synthesized in a single process, whereby the components could be regarded as one and the same entity. For example, hyaluronic acid may be synthesized to include a polyglutamine at one end for linking the polypeptide via transglutaminase.

Specific examples of covalent bonds include those wherein bifunctional cross-linker molecules are used. The cross-linker molecules may be homo-bifunctional or hetero-bifunctional, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl) suberate, bis[2-(succinimidooxycarbonyloxy)ethyl]sulfone, disuccinimidyl suberate, disuccinimidyl tartarate, dimethyl adipimate•2 HCl, dimethyl pimelimidate•2 HCl, dimethyl suberimidate•2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide•2 HCl, 4-(4-N-maleimidophenyl)-butyric acid hydrazide•2 HCl, and 3-[2-pyridyldithio]propionyl hydrazide. The cross-linkers are bis-[β-4-azidosalicylamido)ethyl]disulfide and glutaraldehyde. Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Anine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Other linkers for conjugating hyaluronic acid to the transglutaminase substrates of the invention include peptide linkers described in U.S. Pat. No. 5,342,770. Other chemical linker compositions are described in U.S. Pat. No. 6,303,555 B1 (e.g., carboxylic acids having 4-6 carbon atoms, or ethoxylated polyhydric alcohol, or polyvinyl pyrrolidone, or polyethylene glycol of MW 6000-10,000), U.S. Pat. No. 5,952,454 (spacer for conjugating glycosyl donor to an amine-containing carrier), U.S. Pat. No. 6,361,777 B1 (amino thiol linker), U.S. Pat. No. 4,680,338 (bifunctional linker), U.S. Pat. No. 5,034,514; among others.

In some embodiments, it may be desirable to attach hyaluronic acid to the linking molecule by a bond that cleaves under normal physiological conditions or that can be caused to cleave specifically upon application of a stimulus such as light, whereby the agent can be released. In certain embodiments, hyaluronic acid may be inactive in its conjugated form and active only when released. In other instances, hyaluronic acid would be released to exert an activity remote from its point of attachment to the body tissue. In still other instances, hyaluronic acid would be released in a sustained fashion, to prolong its release as compared to hyaluronic acid applied to tissue but not covalently coupled thereto. Readily cleavable bonds include readily hydrolyzable bonds, for example, ester bonds, amide bonds and Schiff's base-type bonds. Bonds which are cleavable by light are well known. In still other embodiments, the cleavable bond can itself be the peptide bond between monomer units of the linking molecule. Such bonds can be cleaved by proteinases such as trypsin, which can be used in varying strength solutions depending upon the affected tissue.

Noncovalent methods of conjugation may also be used. Noncovalent conjugation includes hydrophobic interactions, ionic interactions, high affinity interactions such as biotin-avidin and biotin-streptavidin complexation and other affinity interactions. In one embodiment, a molecule such as avidin is attached to a linking molecule such as polyglutamine. This conjugate, once attached to tissue according to the invention, then becomes a universal linking moiety for any agent attached to a biotin molecule.

The linking molecules may be part of a microparticle such as a microsphere or a nanosphere, and hyaluronic acid may be contained in the microparticle, either physically entrapped therein, covalently bonded thereto or otherwise physiochemically attached to the microparticle. In preferred embodiments, the microspheres or nanospheres carry, at least on their surface, polymers rich in glutamine, lysine, or both glutamine and lysine. The methods for manufacturing microparticles are documented and do not form a basis for the present invention. The present invention differs from those of the prior art only in that either the polymers of the microparticle structure themselves contain or are derivatized to contain glutamines and/or lysines, or polymers of glutamine, lysine or glutamine and lysine are included within the mixture of polymers forming the matrix, whereby such polymers are entrapped throughout and/or at the surface of the microparticles. Examples of microspheres and nanospheres and their method of manufacture may be found in U.S. Pat. No. 5,075,019, PCT WO95/24929, PCT WO94/23738 and PCT/US96/11990, the disclosures of which are incorporated herein by reference.

In still another aspect of the invention, conjugates of hyaluronic acid with any number of linker molecules, including those recited herein and those known in the art are used for the delivery of a variety of therapeutic agents to a body tissue or surface. In some important embodiments, the linkers are the aliphatic amine and carboxamide containing linkers recited herein. In this aspect of the invention, the hyaluronic acid is intended to act as a carrier molecule for the therapeutic agent, and the hyaluronic acid itself may or may not impart therapeutic benefit. Other linker molecules are disclosed in U.S. Pat. No. 6,267,957 B1. The entire contents of this patent are hereby incorporated by reference. This latter patent also discloses a variety of therapeutic agents that are can be administered via hyaluronic acid. Examples of therapeutic agents that can be used include adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunct to; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LNRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; treatment of amyotrophic lateral sclerosis; treatment of cerebral ischemia; treatment of Paget's disease; treatment of unstable angina; uricosuric; vasoconstrictor; vasodilator; vulnerary; wound healing agent; xanthine oxidase inhibitor.

As will be apparent to one of ordinary skill in the art, these latter conjugates in which hyaluronic acid is used as a carrier for other therapeutic agents can be used in therapeutic or prophylactic methods for subjects in need of such therapeutics. It is within the realm of the medical practitioner to identify subjects that would benefit from administration of such agents.

The compounds of the invention can be used in a number of methods for treating subjects having or at risk of having particular disorders or symptoms, as described herein. A subject having such disorders is one that has been diagnosed, either by a medical practitioner or by self-diagnosis, as having a disorder. Such diagnosis can be made on the basis of symptoms the subject is experiencing or on the basis of laboratory tests. A subject at risk of having a disorder is one that may be predisposed to developing the disorder because of environmental, behavioral or genetic factors. The disorder or condition is treated in a subject having a disorder, while it is prevented in a subject at risk of having a disorder.

The compounds can be used in the treatment or prevention of a number of disorders, including those characterized by dryness. A condition characterized by dryness is one in which a subject experiences a lack of moisture or lubrication at a tissue or body tissue, such as the eye or the mouth. It is to be understood that conditions characterized by dryness can affect any region of the body, both internally and externally. Examples of conditions that can be treated using the methods provided herein include dry eye, dry mouth, dry skin (e.g., wrinkles), dryness in the vaginal cavity, etc. The compounds can be used in the treatment and/or prevention of any disorder for which hyaluronic acid has previously been reported to be therapeutically or prophylactically beneficial. Such disorders have been described before and are known to general medical practitioners.

Skin disorders that can be treated using the compositions described herein include bed sores, trophic ulcers, burns, indolent wounds, post-traumatic ulcers, varicose and postphlebitic ulcers, radionecroses, skin lesions such as those induced by Herpes simplex virus and skin grafts. In these embodiments, the compositions may be administered using gauze pads, cream, sprays, and may include an emulsifying agent. Other agents that may be included in topical formulations include mannitol, polyethylene glycol, oleic acid, glycerol, sorbitol, p-oxymethylbenzoate, paraffin jelly, and glycine.

Other disorders that can be treated using the compounds of the invention include respiratory disorders such as emphysema, chronic bronchitis, asthma, pulmonary edema, acute respiratory distress syndrome, bronchopulmonary dysplasia, pulmonary fibrosis, and pulmonary atelectasis. For these disorders, the compositions may be administered intratracheally, including by way of aerosol, nebulizer, or instillation.

Interstitial cystitis is another disorder that can be treated using the compositions of the invention. Subjects having interstitial cystitis commonly have symptoms such as urgency and increased frequency of urination, suprapubic pain that can be relieved by urination, arthritis, spastic colon and low grade fever. The compositions are preferably instilled directly into the urinary bladder and/or associated anatomical structures, for example using a catheter. The instilled volumes can range from 5 ml to 100 ml, with more preferable volumes between 20 ml and 70 ml. Transabdominal administration is also envisioned.

The compounds can also be used in subjects experiencing joint discomfort. A subject experiencing joint discomfort is one that experiences discomfort or pain in the joints, such as the knee and arm joints. This discomfort may be most often experienced during movement requiring bending of joints, and is associated with a lack of mobility in such joints. It is often a manifestation of arthritis. Subjects to be treated in this manner include those having or at risk of developing joint disorders such as osteoarthritis, acute or chronic synoritis, degenerative processes in articular cartilage, and dry joint disease. The symptoms commonly associated with these conditions include pain and impaired joint function. In these embodiments, the compositions can be administered as intra-articular injections. Such compositions may comprise collagens, proteoglycans, glycosaminoglycans, glycoproteins, sulphated ash, albumin, and preservatives such as sodium benzoate, methyl paraben, propyl paraben. Injection may occur into any joint of the body including but not limited to the carpel, fetlock, coffin, or tibotarsal joints, intertarsal or tarsometatarsal joints, lateral or medial sacs of the femorotibial joint, or femoropatellar joint. Methods relating to the treatment or prevention of joint disorders are particularly suited for animals such as race horses.

The compounds are also useful in subjects having excessive blood clotting or subjects at an elevated risk of developing a blood clot, or having or at risk of having an adverse cardiovascular event. These subjects either have a pre-existing blood clot, or they are predisposed to developing a blood clot due to environmental, behavior (e.g., diet) or genetic factors. An elevated risk of developing a blood clot is one that is above the risk of a normal population of subjects. Excessive blood clotting is blood clotting that occurs inappropriately, and which is either more frequent or more severe than that experienced by a normal population of subjects. These subjects commonly are administered with compounds of the invention using intravenous or intraarterial medical devices such as stents or balloon angioplasties. The compounds may be coated onto the medical device or it may be administered by bolus or continuous injection. The compositions provided herein therefore have utility in the prevention of restenosis. Alternatively, the conjugates optionally together with free hyaluronic acid may be used in the preparation of guide channels, bypasses, artificial veins, shunts, as well as other biomaterials used in the cardiovascular system.

In some embodiments, the compositions are supplied to a body tissue or surface in preparation for treatment with another therapeutic agent. Hyaluronic acid is known to permeabilize tissues and thus is useful for increasing the receptivity of a body tissue for another agent. The tissues so treated may be those that are underperfused either normally or due to a pathological state.

The compositions provided herein can be used in the treatment of subjects having dry eye or dry mouth, but their use is not so limited. Dry eye can result from a number of underlying conditions including but not limited to autoimmune disorders that damage lacrimal (i.e., tear-producing) glands, such as rheumatoid arthritis, Sjögren's syndrome, and systemic lupus erythrematosus, and systemic sclerosis and sarcoidosis. Many of these subjects have decreased tear forming ability. Subjects diagnosed with persistent dryness of the eye including the cornea and/or conjunctiva are suitable candidates for the treatment methods described herein. These subjects may generally complain of mild discomfort to severe pain in the eye, blurred vision, grittiness and/or burning sensation, and itchiness, and may present with corneal ulcers and/or scarring.

The invention also provides methods for treating subjects having dry mouth. Dryness of the mouth (i.e., oral cavity) can result from stress, an underlying condition such as Sjögren's syndrome and systemic sclerosis, infection of the salivary (i.e., saliva-producing) gland, use of particular medications such as anticholinergics, diuretics, antihistamines, clonidine, levodopa, methyldopa, and tricyclic antidepressants, or exposure to radiation therapy. Subjects having dry mouth generally complain of difficulty and soreness in swallowing and speaking, interference with taste sensation, and in some instances, tooth decay. Dry eye is also common in post-menopausal women due to hormonal changes.

In addition to the alleviation of dry eye and dry mouth symptoms, the hyaluronic acid conjugates of the invention can also be used in other situations where it is desirable to maintain a certain level of humidity and moisture in a tissue or surface. Examples include intraocular surgeries such as cataract removal, intraocular lens implantation and keratoplasty.

The compositions can be further used to alleviate symptoms involving dryness of other mucosal tissues (e.g., vaginal, rectal, nasal, anal, etc.) as well as external tissues (e.g., hair, nails, lips, etc.).

In still other embodiments, the conjugates are intended for use in subjects at risk of abnormal platelet clotting since hyaluronic acid has been reported to inhibit platelet aggregation. Such subjects may those undergoing invasive procedures such as stent placements or balloon angiography, and the conjugates of the invention may be administered using these devices, although they administration is not so limited.

It is to be understood that the compositions provided can be used in both therapeutic methods as well as prophylactic methods. When used therapeutically, the conjugates are intended to alleviate pre-existing symptoms in a subject and thus can be administered after the subject complains of the symptoms. When used prophylactically, the conjugates are intended to prevent symptoms from arising, or to delay their onset, in subjects that are engaging, or will engage in activities that are known to cause such symptoms. These activities include in the case of dry eye for example excessive reading, excessive computer use, and potentially even extended use of contact lenses.

A “subject” shall mean a human or vertebrate animal including but not limited to a dog, cat, horse, cow, pig, sheep, goat, chicken, primate, e.g., monkey, fish (aquaculture species), e.g. salmon, rat, and mouse.

The compositions of the invention are administered in effective amounts. The term “effective amount” refers to the amount necessary or sufficient to realize a desired biologic effect. For example, an effective amount of a hyaluronic acid containing conjugate is that amount necessary to reduce or eliminate dry eye symptoms, if the subjects are being treated for such symptoms. If the subject being treated is one having or suspected of having dry mouth, the effective amount is that amount necessary to reduce or eliminate the dry mouth symptoms. As used herein, the term “treat” refers to a reduction or complete elimination of symptoms, such as but not limited to those associated with dry eye or dry mouth. As an example, a subject experiencing dry eye symptoms prior to treatment would be “treated” if the dry eye symptoms diminished in severity or frequency, or were completely eliminated following treatment. Symptoms associated with dry eye or dry mouth are as described herein.

Combined with the teachings provided herein, by choosing among the various conjugate structures and weighing factors such as potency, relative bioavailability, patient body weight, severity of adverse side-effects and preferred mode of administration, an effective prophylactic or therapeutic treatment regimen can be planned which does not cause substantial toxicity or irritation, and yet is entirely effective to treat the particular subject.

The effective amount for any particular application can vary depending on such factors as the disease or condition being treated or the symptoms being alleviated, the particular conjugate being administered, the size of the subject, or the severity of the disease, condition, or symptom. One of ordinary skill in the art can empirically determine the effective amount of a particular conjugate without necessitating undue experimentation.

The effective amount of conjugate will also depend upon the exact nature of the conjugate, including but not limited to the ratio of hyaluronic acid to linking molecule, and the length or molecular weight of the hyaluronic acid and the linking molecule. When applied in a free form, sodium hyaluronate has been administered to the eye in a 0.1% solution (w/v) (i.e., 1 mg/ml). However, given the ability of the hyaluronic acid of the present invention to attach to the ocular surface via the linking molecule, it is expected that a lower amount of hyaluronic acid is necessary in the present formulation. Preferably, the dosage form administers an amount of conjugate having a potency the equivalent of a 0.1% solution of free hyaluronic acid per dose. It is generally recommended that the conjugate be formulated to deliver at least 50 μg of hyaluronic acid per administration if to the eye, and at least 1 mg of hyaluronic acid per administration if to the oral cavity. Formulations intended for the oral cavity may be more concentrated than those for the eye given the larger surface area to be treated in the oral cavity.

Subject doses of the compounds described herein typically range from about 0.001 mg/day to 16,000 mg/day, more typically from about 0.05 mg/day to 8000 mg/day, and most typically from about 0.1 mg/day to 4000 mg/day. Stated in terms of subject body weight (and assuming an average body weight of 80 kg), typical dosages range from about 0.00001 to 200 mg/kg/day, more typically from about 0.0006 to 100 mg/kg/day, and most typically from about 0.001 to 50 mg/kg/day.

Similarly, the volumes in which the conjugates will be delivered will vary depending upon the site of administration. If delivered to the eye, then volumes of less than 2 ml, less than 1 ml, less than 0.5 ml, less than 0.25 ml, less than 0.1 ml, less than 0.05 ml, less than 0.025 ml, or lower are preferable. If delivered to the oral cavity, then volumes may be greater, particularly if the conjugate is delivered in a large volume formulation such as a mouthwash. Alternatively, if the conjugate is delivered as a spray to the oral cavity, then the volume could be on the scale of ocular administration volumes.

The conjugates may be administered to a subject by any mode, however it is preferred that the mode relate to the condition and symptom being treated. For example, when used to treat dry eye symptoms, the conjugates are administered to the eye. When used to treat dry mouth symptoms, the conjugates are administered to the oral cavity. When administered orally, the conjugates are intended to be delivered directly to the oral cavity (including, in some instances, the throat region), rather than the stomach or other regions of the gastrointestinal tract. Other routes of administration include but are not limited to intranasal, intratracheal, inhalation, vaginal, rectal, topical, intra joint, and intravenous.

The compounds will be provided in different vessels, vehicles or formulations depending upon the disorder and mode of administration. For example, and as described in greater detail herein, for oral application, the compounds can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for intra joint application, as an injectable solution delivered intra-articularly, etc.; for blood vessel application, as a coating on a medical device, an injectable solution, etc.; for vaginal or rectal application, as an ointment, a tampon, a suppository, a mucoadhesive formulation, etc.

The conjugates of the invention can be administered to subjects having dry eye in a variety of compositions and physical forms that are suitable for ocular administration. The compositions intended for ocular administration must be compatible with the eye environment, at least in terms of pH, and salt composition and concentration. These compositions should not irritate the eye.

Compositions can be administered to the eye in various physical forms including but not limited to a liquid solution, an ophthalmic ointment or gel, or eye pack such as a cotton pledget. Liquid solutions are conveniently administered with the aid of an eye dropper and may be provided in an eye dropper bottle.

An eye dropper bottle is a container including an eye dropper which is used to remove liquid from the container. It can be glass or plastic, and may be of varying size depending upon the volume of liquid and its shelf life. Solutions that do not contain preservatives, such as ophthalmic preservatives, tend to have a shorter shelf life and thus are generally prepared in smaller volumes. Thus, in some important embodiments, the compositions are provided in eye dropper bottles that contain at a maximum, volumes on the order of 0.5 ml, or volumes on the order of 5.0 ml. These latter embodiments correspond to single use, or single week units, and optionally they do not contain ophthalmic preservatives. A plurality of such small volume bottles (e.g., vials prepared by the blow-fill-seal method) can be provided in a kit, that can optionally comprise an outer housing such as a box or bag, or a backing such as a cardboard or plastic backing. The kit can contain instructions for use of the composition, as outlined herein.

The compositions can also be provided in solutions routinely used and commercially available for eye care. For example, the compositions may be mixed in with contact lens solutions, such as contact lens cleaning solutions, contact lens storage solution, or eye drop solutions for contact lens wearers. Contact lens solutions are known in the art and generally refer to solutions that are used to either store or clean contact lenses, or solutions used by contact lens wearers such as eye drops or artificial tear formulations. When provided together to contact lens wearers, the compositions can reduce friction between the cornea and the contact lens. The compositions can also be provided in the form of films, and such films can be coated onto contact lenses, for example, by contact lens manufacturers, in order to prolong use of contact lens without dry eye or eye irritation. Similarly, the compositions can be included in the solution in which contact lenses are provided commercially.

The compositions can similarly be formulated as ocular gels or ointments, such as those known in the art.

Compositions intended for ocular administration may contain other agents that have been described for ocular solutions, gels, etc. or that are known to be present in tears. An example is lysozyme which is known to be present in tears.

In some embodiments involving ocular administration, the composition may be treated in order to eliminate color (thus rendering the solution clear and colorless). Alternatively, it may be desirable to add or change the color of the composition, particularly if color is used to confirm delivery of the composition to the eye.

In some embodiments, the ocular compositions do not contain preservatives, and rather are sterile filtered (e.g., through a 0.22 μm filter) and packaged as single use amounts. Thus, in some instances, the compositions of the invention are prepared and/or packaged in unit of use amounts. A unit of use amount may be that amount that is required for one administration, or administrations for one day, one week, one month, or longer. Preferably, a unit of unit amount will be that amount required for either one administration or for at most several days (but less than a week) of administration. Unit of use packaging is useful for preventing contamination of solutions, as it reduces the number of times an individual must contact the solution.

The conjugates of the invention can similarly be administered to subjects having dry mouth in a variety of compositions and physical forms suitable for oral or buccal administration. The terms “oral” and “buccal” are used interchangeably herein to indicate the oral cavity, encompassing the lips, teeth, mouth, tongue, palate, and upper throat region. The compositions intended for oral or buccal administration must be compatible with the environment of the oral cavity. The requirements for oral or buccal delivery formulations are generally less strict than those for ocular delivery formulations. However, taste and odor considerations are important in oral or buccal formulations and are most probably less important for ocular formulations.

In preferred embodiments, compositions are delivered to and remain in the oral cavity, regardless of their physical form. Thus, it is preferable that the compositions are provided in forms such as lozenges, sprays, gums, sublingual tablets, mouthwashes, oral gels, toothpastes, mucoadhesive patches, and the like, that remain in the oral cavity and are not ingested into the gastrointestinal tract.

When delivered orally, the conjugates contact the oral mucosa including the sublingual mucosa. “Mucosa” refers to a mucous membrane. “Oral mucosa” as used herein refers to the mucosa of the mouth and upper throat region. “Sublingual” refers to the area of the oral cavity below the tongue.

One suitable oral form is a sublingual tablet. A sublingual tablet delivers the conjugate to the sublingual mucosa. As used herein, “tablet” refers to pharmaceutical dosage forms prepared by compressing or molding. Sublingual tablets are small and flat, for placement under the tongue and designed for rapid, almost instantaneous disintegration and release of conjugate to the sublingual mucosa. The term “disintegration” means breaking apart. Preferably, the sublingual tablets of the present invention disintegrate, to release the conjugate, within five minutes and, more preferably, within a two minute period of time. The released conjugate is then available to be bound to the oral mucosa via the action of endogenous transglutaminase present in the oral cavity.

Other forms of oral delivery formulations include lozenges, gums, and thin dissolvable films.

Oral formulations can also be in liquid form. The liquid can be administered as a spray or drops to the entire oral cavity including to select regions such as the sublingual area. The sprays and drops of the present invention can be administered by means of standard spray bottles or dropper bottles adapted for oral or sublingual administration. The liquid formulation is preferably held in a spray bottle, fine nebulizer, or aerosol mist container, for ease of administration to the oral cavity. Liquid formulations may be held in a dropper or spray bottle calibrated to deliver a predetermined amount of the composition to the oral cavity. Bottles with calibrated sprays or droppers are known in the art.

The conjugates of the invention can also be formulated as oral gels. As an example, the conjugate may be administered in a mucosally adherent, non-water soluble gel. The gel is made from at least one water-insoluble alkyl cellulose or hydroxyalkyl cellulose, a volatile nonaqueous solvent, and the conjugate. Although a bioadhesive polymer may be added, it is not essential. Once the gel is contacted to a mucosal surface, it forms an adhesive film due primarily to the evaporation of the volatile or non-aqueous solvent. The ability of the gel to remain at a mucosal surface is related to its filmy consistency and the presence of non-soluble components. The gel can be applied to the mucosal surface by spraying, dipping, or direct application by finger or swab.

The conjugates of the invention can also be formulated as mouthwashes or toothpastes.

Where necessary, delivery formulations may comprise flavoring, coloring and/or scenting agents. Flavoring, coloring and/or scenting agents help to improve user acceptance of the composition.

Flavoring agents are agents that provide a taste to an otherwise tasteless formulation, agents that enhance a pre-existing but weak taste, or agents that mask or change a pre-existing and unpalatable taste to one that is more palatable. Flavoring agents are known in the art and are commercially available from a number of suppliers such as Warner-Jenkinson Company, Inc. Examples of flavoring agents include peppermint extract, leaf power or oil; spearmint extract, leaf powder or oil; wintergreen oil; vanilla extract; parsley; oregano oil; bay leaf oil; clove oil; sage oil; sassafras oil; lemon oil; orange oil; anise oil; benzaldehyde; almond oil; camphor; cedar leaf oil; marjoram oil; cintronella oil; lavender oil; mustard oil; pine oil; pine needle oil; rosemary oil; thyme oil; cinnamon leaf oil; menthol; carvone; anethole; eugenol; methyl salicylate; limonene; cymene; n-decyl alcohol; citronellol; α-terpineol; methyl acetate; citronellyl acetate; methyl eugenol; cineole; linalool; eyktl linalool; vanillin; thymol; pellira oil; gaultheria oil; eucalyptus oil; caffeine, cream of tartar, lactic acid, malic acid, monosodium glutamate, nitrites, sorbitol, etc. Flavoring agents are most desirable where the formulation is intended for buccal or oral administration. Flavoring agents also include sweetening agents (i.e., sweeteners) such as aspartame, acesulfame, saccharin, dextrose, levulose, sodium cyclamate, stevioside, neo-hesperidyl dihydrochalcone, glycyrrhizin, perillartine, thaumatin, aspartylphenylalanine methyl ester, p-methoxycinnamic aldehyde, etc.

Similarly, coloring agents are agents that provide color to an otherwise colorless formulation, agents that enhance a pre-existing but weak color, or agents that mask or change a pre-existing but potentially unpleasing color. Coloring agents also include agents that convert a colored formulation into a colorless one. Coloring agents are known in the art and can be purchased from the flavoring agent suppliers such as those listed above. Coloring agents may be desirable for ocular as well as oral formulation. An example of a suitable coloring agent is titanium dioxide. Suitable oral formulation coloring agents include FD&C Blue #1, FD&C Yellow #5 and #10, FD&C Red #3 and #40; caramel color or powder (#05439), chocolate shade (#05349), green lake blend (#09236), kowet titanium dioxide (#03970), yellow liquid color (#00403), and nitrites.

Scenting agents are agents that provide scent (i.e., fragrance) to an otherwise odorless formulation, agents that enhance a pre-existing but weak scent, or agents that mask or change a pre-existing but potentially unpleasing odor. Scenting agents also include agents that convert an odored formulation into an odorless one. Scenting agents are known in the art and can be purchased from the flavoring agent suppliers such as those listed above. Examples of scenting agents include natural scenting agents such as extracts of flower, herb, blossom or plant, and artificial scenting agents. Scenting agents may be desirable for ocular as well as oral formulation.

An example of suitable sublingual tablet is made in accordance with the following formulation: hyaluronic acid and polylysine conjugate (formulated to provide 1 mg of conjugate per tablet); mannitol USP (DC grade) 31.5 mg; microcryst, cellulose 40.35 mg; sodium starch glycolate NF 2.6 mg; sodium saccharin, USP 0.5 mg; flavor S.D. peppermint, FCC 0.75 mg; magnasweet MM 188M 0.5 mg; vanilla flavor #800 0.2 mg; D&C Yellow #10, Aluminum Lake 0.2 mg; magnesium stearate, NF 0.5 mg; aerosil 200 0.4 mg.

Another example of a suitable sublingual tablet is made in accordance with the following formulation: hyaluronic acid and polylysine conjugate (formulated to provide 1 mg of conjugate per tablet); mannitol 30.30 mg; microcrystalline cellulose (FMC) 4.00 34.00 mg; sodium starch glycolate (EXPLS TAB Mendell) 2.60; magnesium stearate NF 0.50 mg; sodium saccharin (Mallinckrodt) 2.00 mg; aspartame (Neutrasweet) 4.00 mg; peppermint (Virginia Dare HF82 SD #517) 0.40 mg; vanilla (Virginia Dare 800 NAT) 0.30 mg; MAFCO magnasweet 188M 0.25 mg; prosweet #560 (MM54) 0.75 mg; chocolate flavor #682 2.00 mg; D&C Yellow #10.

Individuals skilled in the art will recognize that modifications to these formulations can be readily made. It is to be understood that other components can be added into the formulations of the invention, including components that are themselves therapeutic or beneficial to the subject. For example, the oral formulations of the invention may include vitamins or fluoride, and the ocular formulations may include therapeutic agents such as anti-glaucoma agents, as are known in the art.

In the above formulations, mannitol, sodium saccharin, peppermint, magnasweet and vanilla are flavoring agents which are capable of masking the taste of the conjugate, or minimally providing a palatable taste. The flavoring agents may be deleted without sacrificing efficacy. However, patient compliance may be more difficult. Flavorings may be altered to suit individual needs and tastes. D&C yellow is used as a colorant. The colorant may be readily deleted or substituted with other dyes. Magnesium stearate and Aerosil-200 are lubricants to release the tablet from press equipment. These ingredients may be substituted or deleted entirely depending on the manufacturing process. Microcrystalline cellulose, mannitol and sodium starch glycolate provide the tablet core. The cellulose and starch facilitate binding the core ingredients and facilitate tablet disintegration in the presence of moisture. The relative amounts of these ingredients may be altered to adjust the disintegration of the tablet.

Quantities of all ingredients are weighed and all the ingredients, other than mannitol and Avicel, are passed through a 80 mesh stainless steel sieve. The materials are blended in a suitably sized polythene bag for about five minutes and transferred to suitable blender, such as a PK Blender. The required quantities of mannitol and Avicel are passed through a 40 mesh stainless steel sieve and added to the PK Blender with the other ingredients. The mixture is blended in the PK Blender for 10 minutes and unloaded. A sample of the blend is subjected to inspection for potency and other quality determining criteria. The bulk density is determined on the blend using bulk density apparatus set for 100 taps. The tablet press is set for the designated punches and the blend is compressed at 80 mg tablet weight.

Tablets are administered by placing a single tablet under the tongue. The tablet is allowed to disintegrate and release the hyaluronic acid containing conjugate which is then attached to the oral mucosa.

Oral solutions are made using distilled sterile and can be made in accordance with the following formulation: hyaluronic acid and polylysine conjugate (formulated to provide 1 mg/ml of conjugate per administration); sodium chloride 0.9%; and benzalkonium chloride 0.1 to 0.2%. The formulation represents an oral solution that can be administered by drops, or in a fine mist, although it is not so limited in its administration route. Individuals skilled in the art will readily recognize that modifications to the formulation can readily be made.

In the formulation above, sodium chloride is used to bring the solution to isotonicity. Such solutions are more comfortable for users; however, sodium chloride may be deleted if desired. Also in the above formulation, benzalkonium chloride is used as a preservative.

In certain embodiments, however, it is preferable to use formulations that are slightly hypertonic, e.g., above 280 mOsm in order to keep the conjugate uncomplexed with itself or with other charged compounds. These embodiments have been described herein.

Oral administration can also be effected using mucoadhesive devices or systems. Preferred systems of this type are those that naturally erode after application. One example of a suitable bioerodible mucoadhesive device or system is BEMA™, which can be formulated as gels, discs or films and can be used at any mucosal surface. Bioerodible mucoadhesive devices are polymer-based systems that allow for the delivery of active agents to mucosal surfaces such as the oral or vaginal mucosa. The bioerodible mucoadhesive device is generally composed of a film that can take a number of forms. In one preferred embodiment, the bioerodible mucoadhesive device is in the form of a small, semi-soft disc. Bioerodible mucoadhesive devices, such as BEMA™ discs, can be impregnated with the conjugate after formation. These devices can adhere to mucosal surfaces, such as the mucosal surfaces of the mouth, vagina, rectum, or anus. As the film bioerodes, the conjugate contained therein is released and attached to the neighboring mucosa. Because the disc essentially dissolves with the moisture of the mucosal surface, there is no requirement that the disc be removed. One advantage of a bioerodible mucoadhesive device is that release of conjugate into surrounding tissues or cavities, without attachment to the mucosa, is minimal. Generally, only one side of the film (e.g., in the disc form) is adhesive to the mucosal surface. For application into the vagina or the anus, it is recommended that the device (e.g., the disc) be rolled and then inserted into the cavity, paying particular attention to the location of the adhesive side of the device.

The bioerodible mucoadhesive device can be specifically synthesized to control residence time of the conjugate in the device, bioerosion kinetics (and thus release time and rate of the active agent), taste of the device (particularly suited for oral administrations), and shape and disc thickness.

Bioerodible mucoadhesive systems and devices are commercially available from Atrix Laboratories (Fort Collins, Colo.), Epic Therapeutics, Inc., Takeda Chemical Industries. Ltd., ALZA Corp., and Alkermes Control Therapeutics, Inc. Reference can be made to U.S. Pat. Nos. 5,650,173; 5,656,297; 5,679,377; 5,800,832; 5,888,533; 5,955,097; 5,962,006; 6,103,266; 6,110,503; 6,156,331; 6,159,498; 6,261,584; 6,265,389; 6,267,981; 6,268,053; 6,275,728; and 6,290,984 among others.

The conjugate may comprise 0.001 to 30% by weight of the device and more preferably between 0.005 to 20% of by weight. Other components may also be present in the BEMA™ disc including plasticizers, flavorings (preferably for oral applications), scenting agents (e.g., fragrances), coloring agents, and preservatives. These latter components can be added to either or both the adhesive and non-adhesive layers of the disc.

The disc may take a variety of shapes or dimensions. The thickness of the disc may vary from 0.05 mm to 1 mm, or 0.1 mm to 5 mm, with either of the adhesive or non-adhesive layers occupying anywhere from 10 to 90% of the overall thickness. As described herein, the conjugate may be prepared for loading into a BEMA™ disc in any of a number of appropriate solvents or solvent combinations including but not limited to water, methanol, ethanol, or low alkyl alcohols such as isopropyl alcohol, alone or in combination.

Disc formation is accomplished using any number of techniques known in the art including but not limited to film dipping, film coating, film casting, spin coating, or spray drying. The two layers of the disc can be formed together or they can be formed separately and then contacted with each other. The disc can be shaped into an ellipse, a square, and a rectangle, but is not so limited.

The compositions of the invention may be administered in pharmaceutically acceptable carriers, which may routinely contain pharmaceutically acceptable concentrations of salts, buffering agents, preservatives, compatible carriers, adjuvants, and optionally other therapeutic ingredients. The term “pharmaceutically acceptable carrier” means one or more compatible solid or liquid fillers, diluents or encapsulating substances, which are suitable for administration to a subject. The term “carrier” denotes an organic and/or inorganic ingredient, natural or synthetic, with which the active ingredient is combined to facilitate application to a subject. The components of the pharmaceutical compositions also are capable of being commingled with the conjugates and compositions of the present invention, and with each other, in a manner in which there is no interaction which would substantially impair desired pharmaceutical efficacy. Pharmaceutically acceptable carriers are known in the art.

The nature of the carrier will vary depending on the site of administration. The carrier must however be suitable for transglutaminase activity. Although transglutaminase tends to function efficiently at a slightly alkaline pH (e.g., pH 6.5 to 9), this may not be suitable particularly for ocular administration. As a result, it is necessary to offset the pH dependency of transglutaminase by increasing salt concentration. It is also necessary to ensure that the hyaluronic acid and linking molecule are not ionically complexed to each other to an extent that renders the linking molecule inaccessible to transglutaminase and/or that precludes hyaluronic acid from functioning effectively as a moisturizer. pH and salt concentration are determined based on maximum transglutaminase activity, minimal ionic interactions between hyaluronic acid and the linking molecule, and minimal irritation of the affected surface or tissue.

Suitable preservatives that are compatible with transglutaminase activity include kathon and methyl paraben. Suitable detergents and/or surfactants that are compatible with transglutaminase activity include hampene led, Tween 20, chemophor RH-40, and DC190. Suitable humectants that are compatible with transglutaminase activity include propylene glycol, butylene glycol, and glucacam E-20. Preservatives that should be avoided include glydant, Dowicil 200, BTC 2125M, and iodoacetamide. Detergents and/or surfactants that should be avoided include Bioterge AS-40, CTAB, monomate CPA 40, and SDS.

Hyaluronic acid as well as other administered compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions may also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules, films and suppositories.

Importantly, the carrier must be suitable for the body tissue or surface which it contacts. As will be known to those of ordinary skill in the art, carriers suitable for ocular administration are required to induce minimal, and preferably, no irritation to the eye. Ocular or ophthalmic formulations are known in the pharmaceutical arts and one of ordinary skill can consult Remington's Pharmaceuticals for guidance as to the composition of such carriers.

Ophthalmic formulations can take the form of liquids such as solutions, emulsions, dispersions, and semisolids such as gels and ointments.

Ophthalmic formulations may or may not contain ophthalmic preservatives. Ophthalmic preservatives are known in the art. Generally, such preservatives are antibiotics, as bacterial infections are one of the most common side effects of administering agents to the eye. Examples of ophthalmic preservatives include organic mercurials (e.g., phenylmercuric nitrate, phenylmercuric acetate, phenylmercuric borate, Thimerosal (Merthiolate®, Lilly)); quaternary ammonium compounds (e.g., benzalkonium chloride), benzethonium chloride, cetyl pyridinium chloride, polyquaternium-1 (POLYQUAD)); parahydroxybenzoic acid esters; and substituted alcohols and phenols (e.g., chlorobutanol, chlorobutanol/phenylethyl alcohol). Other suitable preservatives include methyl paraben and propyl paraben.

The various formulations provided herein may also be sterilized by filtering or heating, as is known in the art.

Ophthalmic formulations can further include isotonicity agents, buffering agents, preservatives (as discussed above), diluents, stabilizers, chelating agents, thickeners, etc. Examples of isotonicity agents include sodium chloride, boric acid, soidum citrate, etc. Examples of buffering agents include borate buffer, phosphate buffer, etc. The pH of ophthalmic formulations should be maintained in the range of 5-8. Examples of diluents include distilled or sterilized water or physiological saline (for aqueous formulations), and vegetable oils, liquid paraffin, mineral oil, propylene glycol, and p-octyldodecanol (for non-aqueous formulations). Examples of stabilizers include sodium sulfite and propylene glycol. An example of a suitable chelating agent is sodium EDTA. Examples of thickeners include glycerol, carboxymethylcellulose, and carboxyvinyl polymer.

Other components that can be included in ophthalmic formulations include sorbic acid, sodium dihydrogen phosphate, sodium borate, sodium hydroxide, potassium chloride, calcium chloride, glycerin, lysozyme, etc.

For oral administration, such carriers enable the compounds of the invention to be formulated as sublingual or buccally absorbed tablets, pills, dragees, capsules, liquids, gels, films, syrups, slurries, suspensions and the like. Oral formulations can also include toothpastes, powders, liquid dentifrice, denture cleansers, mouthwash, denture cleanser, chewing gum, candy, and other foodstuffs. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethyl cellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions, although this is less critical when the conjugate is taken up in the oral cavity than in the gastrointestinal tract. Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

Preservatives such as anti-microbials suitable for oral formulations include thymol, menthol, tricrosan, 4-hexylresorcinol, phenol, eucalyptol, benzoic acid, benzoyl peroxide, butyl paraben, methyl paraben, propyl paraben, salicylamides, etc.

Thickening agents for oral formulations such as toothpastes and the like include carboxyvinyl polymers, carrageenan, hydroxyethyl cellulose, natural gums such as gum karaya, xanthan gum, gum arabic, gum tragacath, etc.

Oral formulations can further contain humectants such as but not limited to glycerin, sorbitol, xylitol, polyethylene glycols, propylene glycols, etc.

If the formulation is a toothpaste or dental cleaner, it may further contain abrasive agents such as silicas such as xerogels, hydrogels, aerogels, calcium or magnesium carbonates, calcium phosphates, alumina and hydrates thereof, aluminosilicates, magnesium and zirconium silicates, etc. These formulations may further comprise fluoride, and anti-calculus agents such as zinc salts, alkali metal pyrophosphates, etc.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Compounds to be administered to the nasal cavity can also be formulated as gels or nasal drops.

For topical administration, the compounds may be provided in any standard formulation that is suitable for the external surface. For example, if the compounds are intended for the skin, they may be provided in an ointment, a lotion, a spray, a gel, a tissue, a wipe (e.g. to treat diaper rash), etc. For application to the lips, the compounds can be provided in a lip balm or lip stick form. As another example, if the compounds are intended for the hair, they may be provided in a spray, a shampoo, a hair fixative such as a hair spray, gel or mousse, etc. For application to the nails, the compounds can be provided in nail polishes and other nail care products.

Although not preferred, in some instances, the compounds may be administered systemically if formulated for such a purpose. Parenteral administration can be performed by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

The compounds may also be formulated in rectal or vaginal compositions such as suppositories (including tampons) or retention enemas, e.g., containing conventional suppository bases such as cocoa butter or other glycerides. Vaginal douche formulation can also be used. Mucosal administration can also be performed using mucoadhesive films such as those described in greater detail herein. In some embodiments involving vaginal administration, it may be desirable to provide the composition in a vehicle that temporarily increases the pH of the vaginal environment in order to facilitate attachment of the conjugate to the vaginal mucosa via vaginal transglutaminase. This increase in pH need not be extended, but rather only long enough to attach an effective amount of conjugate to the tissue. As an example, the pH can be modulated through use of a mucoadhesive disk, a suppository, or a douche solution that provides a local pH of 6.5 to 9.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The compositions of the invention may be administered using sustained release devices, for example a bioerodible mucoadhesive system, such as described herein, as well as those known in the art.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides.

Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Sustained release compositions can be applied topically for example as a gel, an ointment, a cream, or a patch (e.g., a transdermal patch or a mucosal patch, such as a BEMA™ disc). As an example, sustained release biodegradable particles can be applied to the body surface alone or in the context of an ointment, gel or cream. Topical administration includes administration to a skin surface and a mucosal surface. Mucosal surface delivery can be effected via lipsticks, lip treatments such as lip balms, cold sore ointments; sunscreen ointments; oral gels such as those used for mouth sores (e.g., radiation or chemotherapy induced mouth sores); mouthwashes; toothpaste; inhalants; surface patches; and the like. Alternatively, they can implanted. In preferred embodiments, the sustained release devices are bioerodible or biodegradable. In other preferred embodiments, the sustained release devices are adhesive to the surface to which they are applied (e.g., skin or mucosa), with preferred forms being the bioerodible mucoadhesive (e.g., BEMA™) devices of the invention. The art is familiar with such devices.

The compositions of the invention can be provided in a kit according to some aspects. As used herein, when a composition is provided in a kit, it is intended that the composition is in packaged or contained in a first container (such as a bottle) which is then further packaged in a second container (such as a box, carton, or bag). In either case, it may be desirable to include instructions for use of the compositions. Such instructions can be provided directly on the outer surface label of the first container (i.e., that which directly houses the composition), or on the outer surface label of the second container (i.e., that which houses the first container). Alternatively, the instructions for use may be provided separately from either container, such as for example on a separate sheet of paper provided within the second container. The instructions for use will contain information such as but not limited to the amount to be delivered in a single dose, the maximum amount not to be exceeded for any given interval (for example the maximum daily dose), the method of administering and the site of administration, the subjects to be treated and those not to be treated with the formulations, contraindications, as well as active and inactive ingredients of the formulation.

The following examples are included for purposes of illustration and are not intended to limit the scope of the invention.

### EXAMPLES

### Example 1

### Conjugation of Poly(L-Lysine) with Hyaluronic Acid Via Reductive Amination

Introduction:

Conjugation of polylysine (PLL) to hyaluronic acid (HA) is based on the ability of the aldehyde group of the terminal sugar residue of hyaluronic acid to form a Schiff base with -amino groups of polylysine. The formed Schiff base is not stable and is easily reversed by hydrolysis. A number of reducing agents can be used to convert the Schiff base into a stable secondary amine. The reduction reaction is best facilitated by sodium cyanoborohydrate because of the high reactivity of this reagent toward the Schiff base and low reactivity to the aldehyde group. 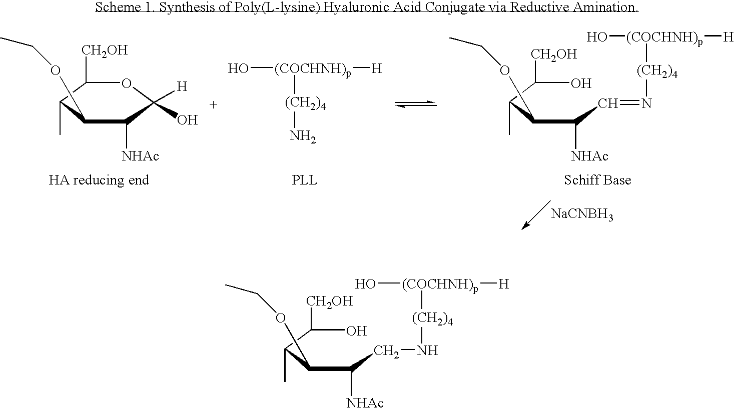
Materials and Methods:

Hyaluronic acid with molecular weight viscosity average of 220,000, in its sodium salt form, was purchased from Lifecore Biomedical (Chaska, Minn.). FITC labeled poly-L-Lysine (i.e., PLL-FITC), with a molecular weight of 15,000-30,000 and degree of substitution of 0.003-0.01 mole FITC per mole lysine monomer, was purchased from Sigma Chemical Co. (St. Louis, Mo.). Sodium cyanoborohydride (NaBH₃CN) was from Aldrich (Milwaukee, Wis.).

PLL was conjugated to HA by reductive amination using NaBH₃CN as a reducing agent, as shown in Scheme 1. HA (100 mg) and PLL (10 mg) were dissolved in 15 ml of sodium borate buffer (0.1M, pH 8.5) containing 1 M NaCl. The concentration of HA was 6 mg/ml, while the concentration of PLL varied from 0.6 to 2.4 mg/ml. Sodium cyanoborohydride was added to the reaction at a concentration 24 mM, approximately 1000 molar excess to the HA reducing ends. Reaction mixtures were incubated at 40° C. under constant stirring, and aliquots were withdrawn from the reactions immediately, 3 days and 6 days after mixing reagents. Aliquots were diluted with phosphate buffer to an HA concentration 3 mg/ml and a PLL concentration 0.3 mg/ml, and analyzed by gel permeation chromatography (GPC). A negative control experiment was conducted under the same conditions except that sodium cyanoborohydride was not added to the reaction. Final products were harvested at 6 days of reaction time for the experiment and 3 days of reaction time for the control, and dialyzed against 0.5 M NaCl aqueous solution using a Spectra/Por 7 membrane (molecular weight cutoff of 50,000) to remove unreacted sodium cyanoborohydride. For the experiments conducted at low pH, samples were diluted by acetic acid buffer solution (50 mM acetic acid, 0.2 M Na₂SO₄) to pH 3.5, stored one day and analyzed by gel permeation chromatography (GPC).

GPC was carried out using a Waters pumping system at the flow rate of 1.0 ml/min at 25° C. using an Ultrahydrogel linear column (Waters Corporation, Milford, Mass., US) equipped with a Waters 600 Controller and 717 Autosampler. The aqueous solution containing 0.2 M Na₂SO₄ and 5 mM sodium phosphate buffer (pH 8.0) was used as a mobile phase. Five hundred microliters of each sample were injected into the column. Eluate was detected by a Waters 996 Photodiode Array Detector, processed at 208 nm and 490 nm wavelengths, and a 474 Scanning Fluorescence Detector (with excitation wavelengths of 490 nm and 530 nm). For the experiments at low pH, running and detecting conditions were the same, except that the composition of the eluting buffer was 50 mM acetic acid, 0.2 M Na₂SO₄ at pH 3.5.

Results:

UV absorbance (at 490 nm and 208 μm) and fluorescence (at 490 nm and 530 nm) GPC profiles of the reaction mixture were analyzed at 5 minutes, 3 days and 6 days. A peak with maximum retention time of 7 minutes is attributed to HA while a peak with maximum retention time at 9 minutes is assigned to PLL-FITC. The assignment is based on GPC profiles of the individual HA and PLL-FITC compounds. Transformations during the reactions are traced by the conversion of PLL-FITC, detected by UV absorbance at 490 nm and fluorescence at 530 μm. PLL-FITC has maximum absorbance while HA does not absorb under the above conditions. After the reaction starts, peak area corresponding to PLL-FITC decreases while the area of the peak corresponding to HA (peak maximum at 7 minute retention time) increases. The appearance of the new peak (7 minute retention time) is due to PLL-FITC conjugation to HA. The increase of its absorbance can be attributed to the increase of conjugation between PLL-FITC and HA in the course of the reaction. The degree of conjugation with time is determined by the integration of the corresponding peaks and constitutes 2.4% (5 min), 31.2% (3 days), 37.5% (6 days).

The negative control experiment conducted in the absence of NaBH₃CN was compared with the reaction in the presence of reducing agent. GPC profiles detected by UV absorbance at 490 nm and fluorescence at 530 nm were analyzed. The degree of conjugation in the control experiment is approximately 10%, which is three times less than the degree of conjugation observed in the presence of NaBH₃CN.

PLL-FITC is positively charged while HA is negatively charged at the reaction pH 8.5. Although the conjugation reaction between HA and PLL-FITC was accomplished at high salt concentration (1M NaCl) to prevent ionic complex formation between reagents, the possibility of such complex formation remains. In order to distinguish between ionic and covalent bond formation the reaction mixture was acidified with acetic acid to pH 3.5. Under acidic conditions, HA becomes protonized and if an ionic complex was formed it would be unstable. In contrast, the chemical covalent bond is stable under the same conditions. Negative control, and test reaction mixtures obtained with NaBH₃CN, were stored at pH 3.5 for one day and analyzed by GPC (acetic buffer, 50 Mm, 0.2 M Na₂SO₄). The fraction of the peak assigned to conjugate is the same as before acidification: 10% for control and 34% for the experiment with NaBH₃CN. The HA-PLL-FITC conjugate formed by covalent bonding is stable enough not to be destroyed at pH 3.5.

### Example 2

### Linking HA-PLL-FITC to in a Rabbit Eye Model

Materials and Methods:

The extent and duration of attachment of FITC-labeled polylysine and FITC-labeled polylysine conjugated to hyaluronic acid was tested using an in vivo rabbit cornea model. Ten New Zealand White rabbits were used in this randomized, double-masked, placebo controlled, single-centered, contralateral group, pre-clinical study.

Each rabbit eye was randomly assigned to one of four treatments: Active 1 (vehicle plus 0.42% FITC-labeled PCS-101 (hyaluronic acid conjugated to polylysine, including free hyaluronic acid in about a 1:1 molar proportion with conjugate; average molecular weight of HA is 220,000 Da, and of polylysine is 15,000-30,000 Da) (sample size of 9 eyes)); Active 2 (vehicle plus 2% FITC-labeled polylysine) (sample size of 4 eyes)), vehicle (20 mM sodium borate, pH 7.8 plus 80 mM NaCl) (sample size of 5 eyes); and placebo (phosphate-buffered saline (PBS), pH 7.4) (sample size of 2 eyes). The fluorescence intensity in the original solution is 175 fold lower in the PCS-101 solution than in the poly-lysine solution.

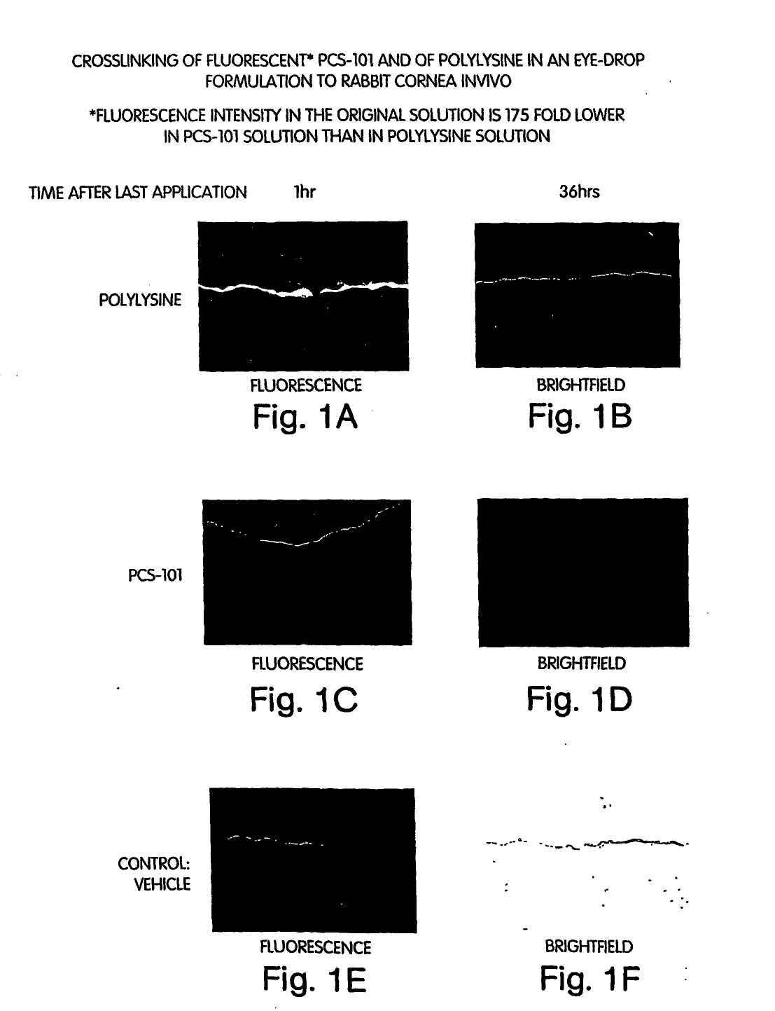

The compositions were administered in equal volumes of 40 μl per administration, twice a day onto the corresponding eye of a live rabbit on each of three consecutive days, for a total of six drops per eye. One hour, sixteen hours, and thirty six hours after the last drop administration, animals were sacrificed, and corneas were removed and frozen in OCT medium for cross-sectioning. Sections were photographed with a Spot RT digital camera (Diagnostic Instrument, Inc.) under FITC and bright field illumination under 40× magnification for histological examination. All rabbits completed the study and were evaluable for all variables. The results are shown in FIGS. 1A-1F which show the crosslinking of fluorescent PCS-101 and poly-lysine in an eye-drop formulation to rabbit cornea in vivo at 1 hour and 36 hours after the last application.

Results:

FIGS. 1A-1F are photographs of rabbit corneal cross-sections featuring fluorescent-labeled TransLink™ system products from the eye test. The top row (FIGS. 1A and 1B) shows the polylysine TransLink™ system anchor with a fluorescein label (no active agent) on the cornea, from rabbits sacrificed one hour and thirty-six hours after the last of six drops administered over three days. It is clearly shown that the Pericor polylysine anchor maintains a remarkable durability throughout the top layer of dead corneal epithelia after an hour, and exhibits presence after thirty-six hours. The reduction in signal between the one hour and thirty six hour timepoints is most likely the result of the natural shedding of the epithelia over 24-36 hours and quenching of the fluorescent molecule used as the marker.

FIGS. 1C and 1D show the attachment of the hyaluronic acid and polylysine conjugate to the rabbit cornea at one hour and thirty six hours. The polylysine in the conjugate is similarly FITC-labeled. Stock solutions of FITC-labeled conjugate exhibited about 175 times less fluorescence intensity compared with stock solutions of FITC-labeled polylysine prior to attachment. The hyaluronic acid containing conjugate cross-links durably to the top layer of epithelia cells of the cornea (and the conjunctiva/lids, data not shown) at the one hour time point. At thirty six hours after the last application, a minute but still detectable amount of conjugate is present on the cornea.

FIG. 1E demonstrates the level of background fluorescence when corneas are treated with vehicle alone. FIG. 1F demonstrates the structure of the cornea on cross section.

Conclusions:

Extent and Duration of Attachment:

Administration of six drops of FITC-conjugated polylysine resulted in durable attachment of polylysine to rabbit cornea for at least thirty six hours following the last drop application. Analysis of the treated eye indicates that the polylysine linking molecule attached to mature surface epithelial cells of the cornea and to conjunctiva and lid as well. This finding was surprising as the surface cells of the eye in normal rabbits have a natural tear film covering that would be expected to preclude attachment of agents thereto. The longevity of the attachment of polylysine indicates that such attachment is resistant to the natural environment of the eye and is not mechanically disrupted by blinking.

Administration of six drops of FITC-labeled polylysine conjugated to hyaluronic acid similarly resulted in attachment of polylysine to rabbit cornea for at least one hour. Although minimal amounts of labeled polylysine were detectable at thirty six hours, this may be partly explained by the lower fluorescence intensity of the stock solution of fluorescently labeled conjugate. Consistent with the results for polylysine alone, durable attachment of the hyaluronic acid conjugate to the ocular surface was achieved. Importantly, no eye irritation was observed.

Irritation:

Active 1 induced a statistically significant increase in hyperemia over baseline at only one time point, 5 min after first application only. However, this change of less than a half grade is not considered clinically significant for attaining an irritation reaction in this model. Active 2 induced statistically significant increases in hyperemia over baseline at four time points. In addition, Active 1 did not induce any statistically significant increases in composite ocular irritation over baseline, whereas Active 2 induced statistically significant increases in composite ocular irritation over baseline at four time points.

There was no statistically significant differences in hyperemia or composite ocular irritation between Active 1 and placebo at any time point. However, there were several statistically significant differences between Active 1 and Active 2 over days 1-3, specifically 15 time points for hyperemia and 8 time points for composite irritation.

Active 1 did not produce any clinically significant changes as compared with baseline or the placebo group at any time points. Also at all but one time point, the compound did not produce any statistically significant changes in any of the irritation scores. As a result, Active 1 did not show to be an irritating agent in this study.

### Example 3

### Ability of HA-Polylysine Conjugate to Bind to the Cornified Layer of Human Finger In Vivo

Materials and Methods:

Reaction solutions are 0.34 μg/μl HA-PLL-FITC conjugate (by PLL-FITC content) in 0.1 M glycine buffer 0.15 M NaCl, pH 8 in a total reaction volume of 20 μl

Human finger was rinsed in water and dried, after which the reaction solution was applied. The reaction solution was rubbed onto the skin using a powder free finger cot for 10 seconds, and left to dry at room temperature. The finger was then washed with water and dried. At various time-points after washing (0, 2.5, 6, and 24 hours), the top surface of the finger was photographed under FITC illumination (at 2× magnification) with a Spot RT digital camera (Diagnostic Instrument, Inc.).

Results:

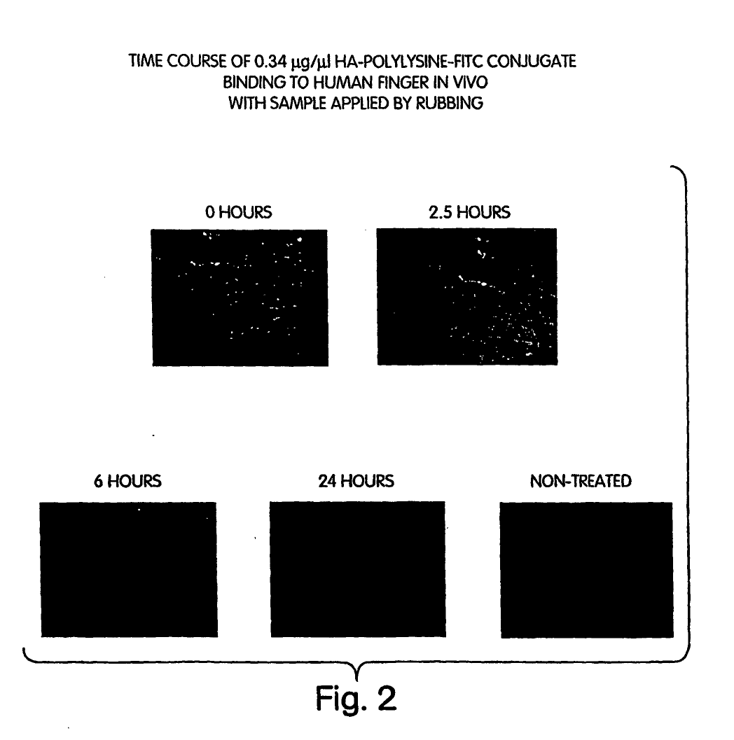

FIG. 2 shows the data as a time course of 0.34 μg/μl of HA-PLL-FITC conjugate binding to human finger in vivo with the sample applied by rubbing. The time course runs from 0 hours, 2.5 hours, 6 hours and 24 hours. Also shown is the level of staining of non-treated tissue (control).

The bright fluorescence observed after washing demonstrates that the HA-PLL conjugate was crosslinked to the cornified layer of human finger in vivo (FIG. 2). Even 24 hours after washing, a significant amount of fluorescence is still visible, showing the durability of crosslinked HA-PLL conjugate on the human skin in vivo over a 24-hour period.

### Example 4

### Uptake after Repeated Applications of PCS-101 without Added Transglutaminase

Materials and Methods:

The complete reaction solution contained 10 mg/ml of PCS-101 (HA-PLL-FITC and free HA) in sterile buffer containing 20 mM sodium borate, pH 7.8 plus 80 mM NaCl. The total reaction volume was 50 μl.

Intact rabbit eyeballs were rinsed in PBS buffer. The reaction solution was applied onto the center of each cornea using a 0.5 cm² cylinder and incubated at 37° C. for 1 minute in a humid chamber. After the incubation time, the reaction solution was removed and the cornea was washed in PBS buffer for one minute at 25° C. Then, one rabbit eye sample was removed while the remaining samples each received an additional application of the reaction solution followed by re-incubation at 37° C. This protocol was repeated until all samples were treated with PCS-101, for a total of 1, 2, 4, 6, 8, 10 and 12 applications. Each cornea was photographed with a Spot RT digital camera (Diagnostic Instrument, Inc.) under FITC illumination with a 2× objective after washing. The cornea was then excised and frozen in OCT medium. Frozen tissue sections were made from each sample and the sections were photographed with a Spot RT digital camera (Diagnostic Instrument, Inc.) under epifluorescence with a 20× objective and appropriate filters

Results:

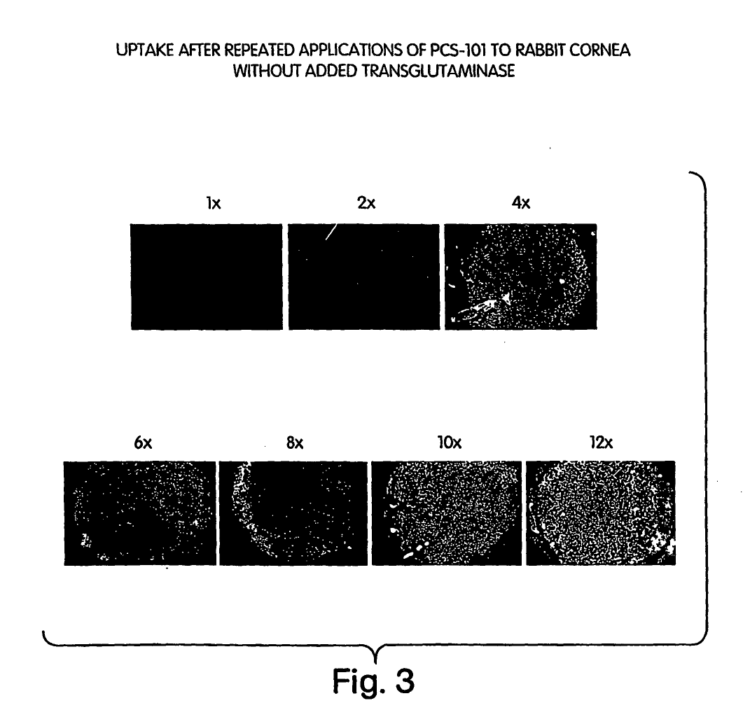

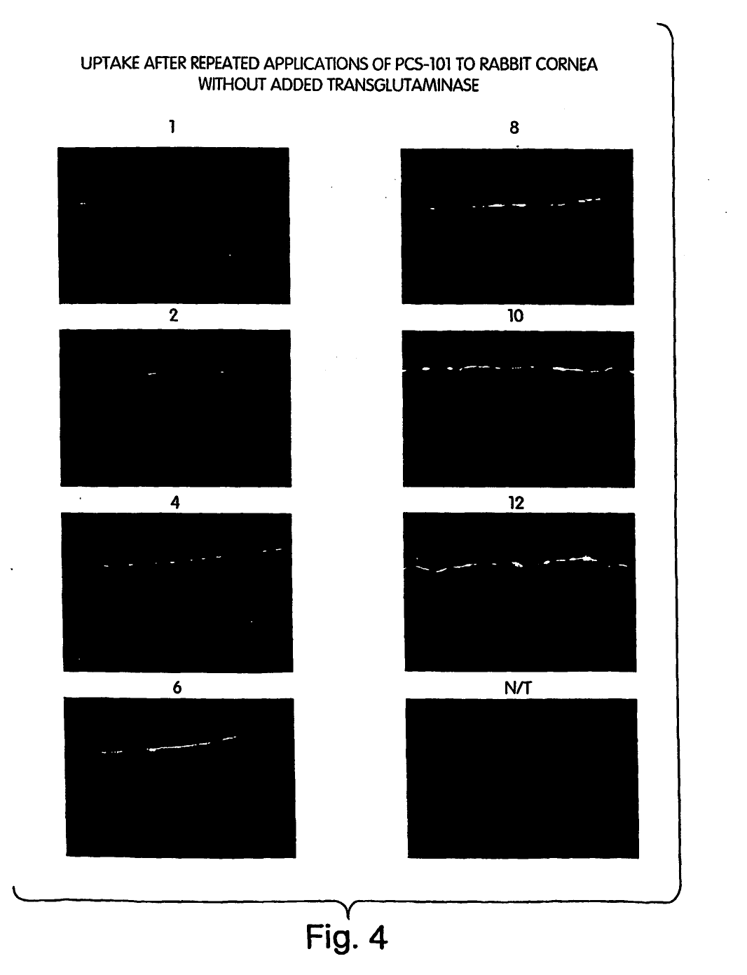

FIGS. 3 and 4 demonstrates the uptake of PCS-101 after repeated applications to rabbit cornea without added transglutaminase. The number of applications is shown and ranges from 1× to 12×. The fluorescence observed after the first application clearly demonstrates that PCS-101-FITC was crosslinked to the cornified layer of rabbit cornea. The amount of PCS-101-FITC retained by rabbit eye cornea appears to increase almost linearly with successive applications of the PCS-101 up to 12 applications (FIGS. 3 and 4). This occurred in the absence of exogenously added transglutaminase and Ca++ in the buffer.

### Example 5

### Coupling of PCS 201 (HA-PLL-FITC Conjugate) to the Pig Palate, Gum and Lower Tongue Epithelia without Added Transglutaminase, and Inhibition by EDTA

Materials and Methods:

The complete reaction solution contained 100 mM glycine (pH 8.2), 150 mM NaCl buffer and 1500 μg of HA-PLL-FITC conjugate. The total reaction volume is 300 μl. The negative control contained 140 mM EDTA in the reaction solution.

Pig palate, gum and lower tongue samples were briefly washed in PBS buffer and incubated in the reaction solutions at 37° C. for one hour. Samples were then washed with PBS Buffer for one hour under agitation. Frozen tissue sections were made and stained with DAPI. The sections were photographed under epi-fluorescence illumination with appropriate filters showing FITC in green and DAPI in blue.

Results:

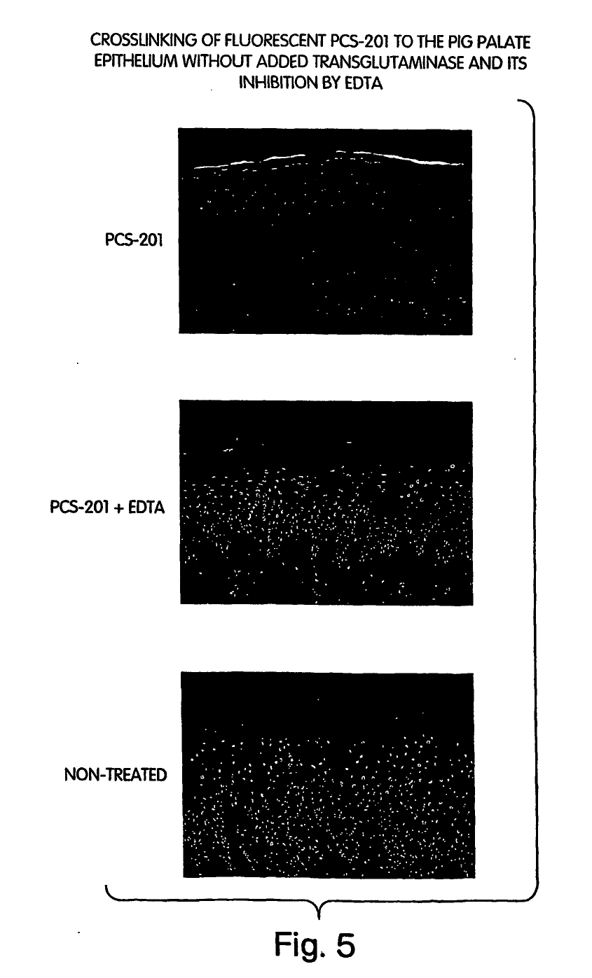

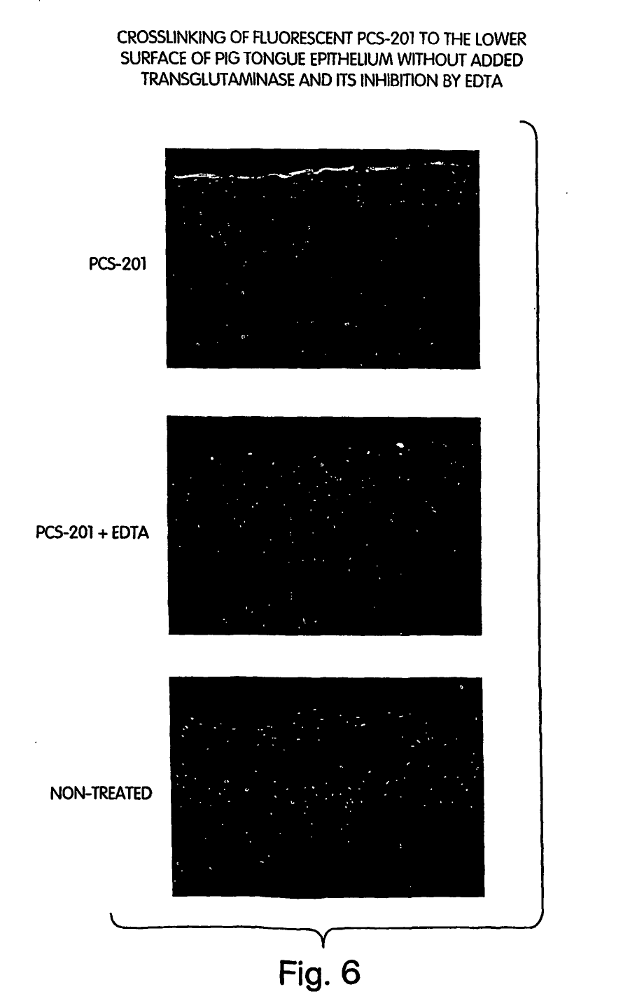

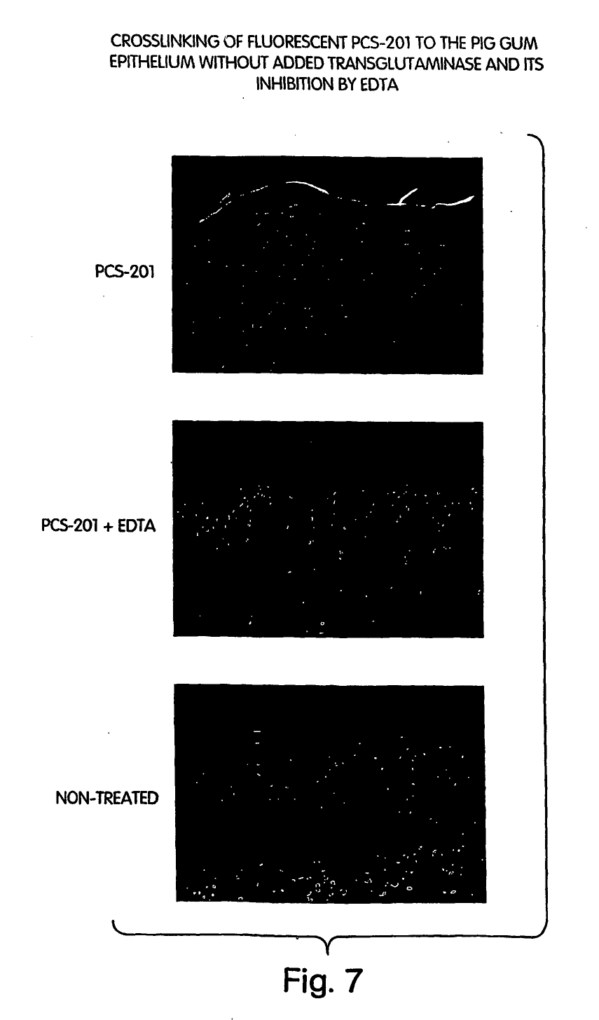

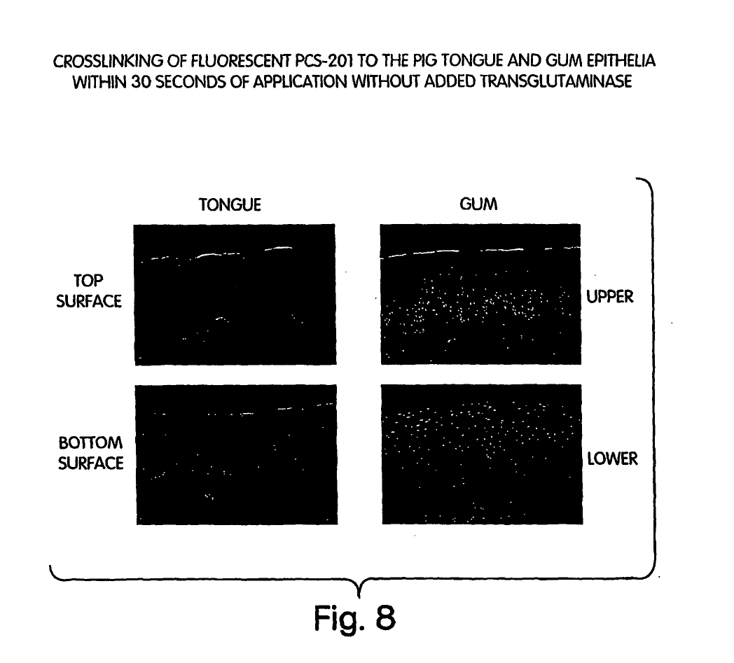

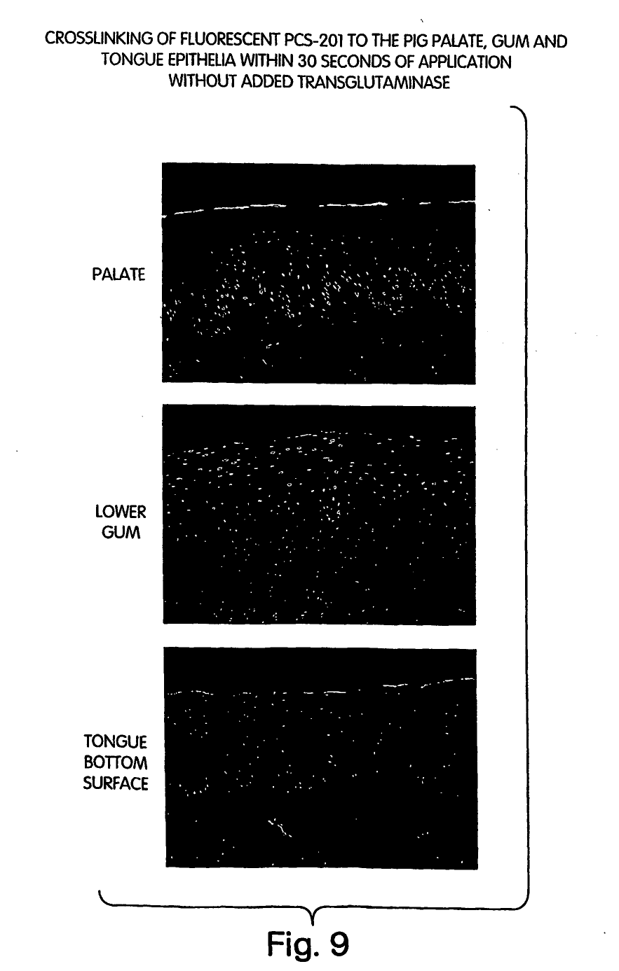

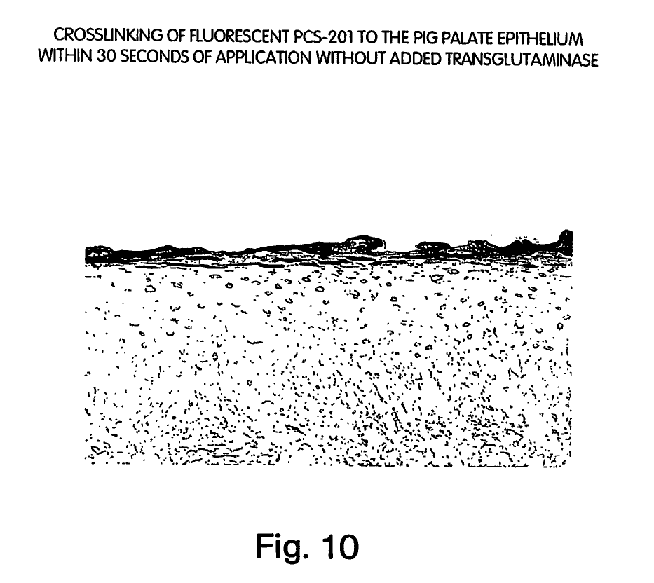

FIG. 5 demonstrates the crosslinking of fluorescent PCS-201 to pig palate epithelium in the absence of exogenous transglutaminase and its inhibition by EDTA. FIG. 6 demonstrates the crosslinking of fluorescent PCS-201 to the lower surface of pig tongue epithelium in the absence of exogenous transglutaminase and its inhibition by EDTA. FIG. 7 demonstrates crosslinking of fluorescent PCS-201 to pig gum epithelium in the absence of exogenous transglutaminase and its inhibition by EDTA. FIG. 8 demonstrates crosslinking of fluorescent PCS-201 to pig tongue and gum epithelium within 30 seconds of application in the absence of exogenous transglutaminase. FIG. 9 demonstrates crosslinking of fluorescent PCS-101 to pig palate, gum and tongue epithelia within 30 seconds of application in the absence of exogenous transglutaminase. FIG. 10 demonstrates crosslinking of fluorescent PCS-201 to pig mouth epithelium within 30 seconds of application in the absence of exogenous transglutaminase. Endogenous transglutaminase crosslinks a significant amount of HA-PLL-FITC conjugate to the cornified layer of the pig palate, gum and lower tongue epithelia. EDTA inhibits the coupling of HA-PLL-FITC conjugate to the cornified layer of the pig palate, gum and lower tongue epithelia (FIGS. 5-10).

### Example 6

### Coupling of PCS-201-FITC and Polylysine to the Inner Lining of Pig Aorta without Added Transglutaminase as a Function of Time

Materials and Methods:

The reaction solutions contained 1 μg/μl of PLL-FITC (MW=24 kD) in 100 mM Glycine, 150 mM NaCl (pH 8.2), and 5 μg/μl of HA-PLL-FITC conjugate in 100 mM Glycine, 150 mM NaCl (pH 8.2) (PCS-201-FITC). Fluorescence intensity in the reaction solution is 17 fold lower in the PCS-201 solution than in PLL-FITC solution. The total reaction volume is 500 μl.

Pig aorta was washed in PBS buffer and, while submerged in the buffer to avoid drying of the tissue, cut into square pieces of 0.5 cm² each. Each piece of aorta was then incubated in the reaction mixture for different time periods (1, 15, 30 and 60 minutes) at 37° C. After a rinse with PBS, samples were washed with PBS for 1 hour under agitation. The aorta samples were then embedded and frozen in OCT compound. The samples were sectioned and photographed under fluorescent illumination.

Results:

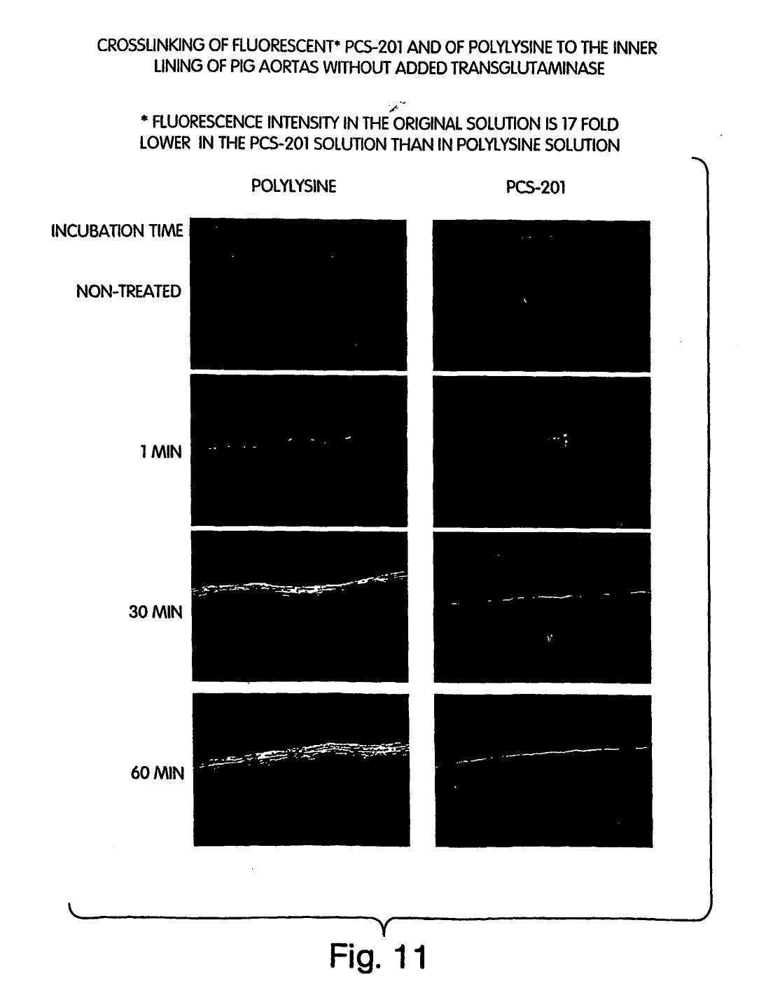

FIG. 11 demonstrates crosslinking of fluorescent PCS-201 and PLL to the inner lining of pig aorta in the absence of exogenous transglutaminase. The fluorescence intensity in the original solution is 17 fold lower in the PCS-201 solution than in the PLL solution. Binding of PLL-FITC and PCS-201 to the inner lining of the aorta was observed after 1 minute of incubation, as indicated by the fluorescence detection. The binding of PLL-FITC and PCS-201 increased with the length of incubation, as indicated by the fluorescence intensity as well as its uniformity on the histological section (FIG. 11). The lower fluorescence intensity of PCS-201 retained by the aorta tissue as compared to that of PLL-FITC is partially attributed to the fact that fluorescence intensity in the reaction solution was 17 fold lower in the PCS-201 solution than in PLL-FITC solution.

### Example 7

### Binding of HA-Polylysine Conjugate to the Cornified Layer of Rabbit Cornea at Different Salt Concentrations

Materials and Methods:

The conjugate stock was made by resuspending lyophilized conjugate in 0.1 M glycine and quantified using fluorescence of FITC (stock concentration=0.1 mg/ml). The reaction groups were 0.1 mg/ml HA-PLL-FITC in 0.2 M glycine, with increasing salt concentrations (0, 50, 150, 300, 500 mM NaCl), and 0.1 mg/ml PLL-FITC in 0.1 M glycine, with increasing salt concentrations (0, 50, 150, 300, 500 mM NaCl). The total reaction volume was 70 μl.

Rabbit intact eyeball was rinsed in PBS buffer. The reaction solution was applied onto the center of the cornea using a 0.5 cm² cloning cylinder and incubated for 1 hour at 37° C. After 1 hour, the eyeball was washed in 30 ml of PBS for 1 hour with agitation. The cornea was photographed with a Spot RT digital camera (Diagnostic Instrument, Inc.) under FITC illumination before and after washing. The cornea was then excised and frozen in OCT medium. FITC fluorescence was quantitated under the following conditions: PLL 2× magnifications, 2 sec exposure, ND4 filter; HA-PLL conjugate: 2× magnification, 2 sec exposure, no filters.

Results:

TABLE 1
Quantification of FITC fluorescence from the topical
view of cornea after washing.

| Sample | Salt Concentration | Maximum Intensity |
|---|---|---|
| HA-PLL | No salt | 737 |
| | 50 mM | 760 |
| | 150 mM | 1394 |
| | 300 mM | 2605 |
| | 500 mM | 3112 |
| PLL | No salt | 2818 |
| | 50 mM | 4095 |
| | 150 mM | 3656 |
| | 300 mM | 3236 |
| | 500 mM | 1664 |

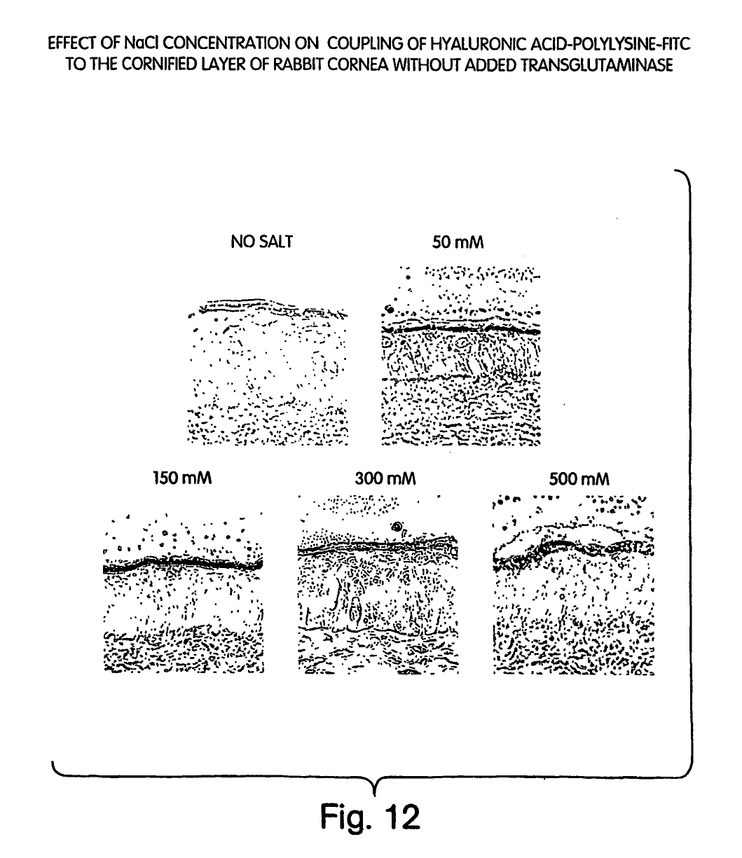

FIG. 12 demonstrates the effect of NaCl concentration on coupling of HA-PLL-FITC to the cornified layer of rabbit cornea in the absence of exogenous transglutaminase. Salt concentration ranged from 0 to 500 mM. Table 1 shows the level of FITC binding to corneal tissues using either PLL or the HA-PLL conjugate. Binding of the HA-PLL conjugate to the cornified layer of rabbit cornea increased steadily with an increase in NaCl concentration, probably as a result of dissociation of any HA and PLL ionic complex, thereby exposing PLL as a substrate for transglutaminase (FIG. 12). Binding of PLL alone decreased steadily with an increase in NaCl concentration, possibly due to changes in the osmotic flow or the effect of the salt on transglutaminase.

### Example 8

### In Vivo Study

Introduction:

Human trials were conducted to determine the effect of the HA-PLL conjugate in post-Lasik™ dry eye patients. The study is designed to compare the efficacy of HA-PLL in post-Lasik patients experiencing dry eye in comparison to normal patients and dry eye patients receiving placebo.

Preliminary Results:

The first patient (patient #1) was given high molecular weight HA, PCS-102 (0.15% of PolyL-810kd mw HA). Patient #1 was operated on 9 months ago, by a different surgeon, and has had extremely painful dry eye ever since. The second patient (patient #2) was given a high concentration of the current molecular weight, PCS-101 (0.4% of PolyL-220kd mw HA). Patient #2 was operated on 3 weeks ago and 1 full week transpired before her treatment (to eliminate any effects of topical steroid use post-surgery). Both patients were experiencing dry eye.

The indicative measures used here are: 1) tear film breakup time (TFBUT); 2) the Schirmer II test (which measures the amount of moisture on the corneal surface by way of a “litmus type strip”); and 3) the presence of flourescence punctuate (classic “staining” endpoint). The second and third measures have been approved by the FDA as acceptable signs of efficacy. Both eyes were evaluated independently, with RE=right eye; LE=left eye.

Each patient was measured before treatment for a baseline TFBUT and Schirmer score. Normal people would have roughly a score of 10 for each of those two measures. Both patients exhibited below average scores on both measures as expected, as well as reporting painful symptoms.

Each patient was initially given only one drop of the respective HA-PLL conjugate and then measured in 15 minute intervals up to 90 minutes (in the case of patient #2).

Each patient was given 6 drops per day for 2 days then asked to stop. They were then both measured in 15 minute intervals up to 90 minutes to see if the treatment improved their TFBUT, Schirmer and flourescent staining score.

Patient #2 was asked to continue the therapy for 1 additional week at only three drops per day (QID). Following this, patient #2 was measured after a 10-12 hour period (overnight) without any additional application of the drops, and then again in 15 minute intervals up to an additional 90 minutes.

Fro patient #2, Schirmer test scores jumped significantly, even 13.5 hours after the last application. TFBUT and staining scores likewise appear to be statistically significant improvements. In addition, the patients reported great relief and a desire to not part with the products.

The data for both patients is provided in Table 2.

### Example 9

### Crosslinking of PCS-101 (HA-FITC Conjugated to PLL-TRITC) after Repeated Applications to Rabbit Cornea without Added Transglutaminase in Comparison to Free HA (HA-FITC)

Material and Methods:

A batch of free HA was first labeled on COOH groups using fluorescein amine and purified. One part of the labeled HA (HA-FITC) was kept as control whereas the remaining was conjugated to polyLysine that has been previously labeled with TRITC and purified. After conjugation, the double-labeled PCS-101 (HA-FITC conjugated to PLL-TRITC) was purified.

For assessment of crosslinking to Rabbit cornea, the reaction groups were

Group I: 10 mg/ml PCS-101 (HA-FITC conjugated to PLL-TRITC) in 20 mM Borate, 80 mM NaCl, pH 7.8, and

Group II: 10 mg/ml free HA (HA-FITC) in 20 mM Borate, 80 mM NaCl, pH 7.8.

The total reaction volume was 50 μl. The FITC fluorescence intensity, viscosity and molecular weight of HA in the applied solutions of PCS-101 and free HA were the same.

Rabbit intact eyeball was rinsed in PBS buffer. The reaction solution was applied onto the center of the cornea using a 0.5 cm² cylinder and incubated for 1 minute at 37° C. The treatment area was then washed for less than 1 minute with 150 μl of 1×PBS within the cylinder. This process of application and washing was repeated 6 times for each eye.

The cornea was then photographed with a Spot RT digital camera (Diagnostic Instrument, Inc.) under FITC illumination. The cornea was then excised and frozen in OCT medium. Histological sections were made and photographed under FITC illumination for quantification of fluorescence. Results:

Results:

TABLE 3
Quantification of FITC fluorescence on histological sections.

| | Corrected Mean FITC | |
|---|---|---|
| | Fluorescence Intensity | Fold Difference |
| HA-FITC-K-TRITC | 256.6503 | 24 |
| HA-FITC | 10.9462 | |
| Non Treated | 0 | |

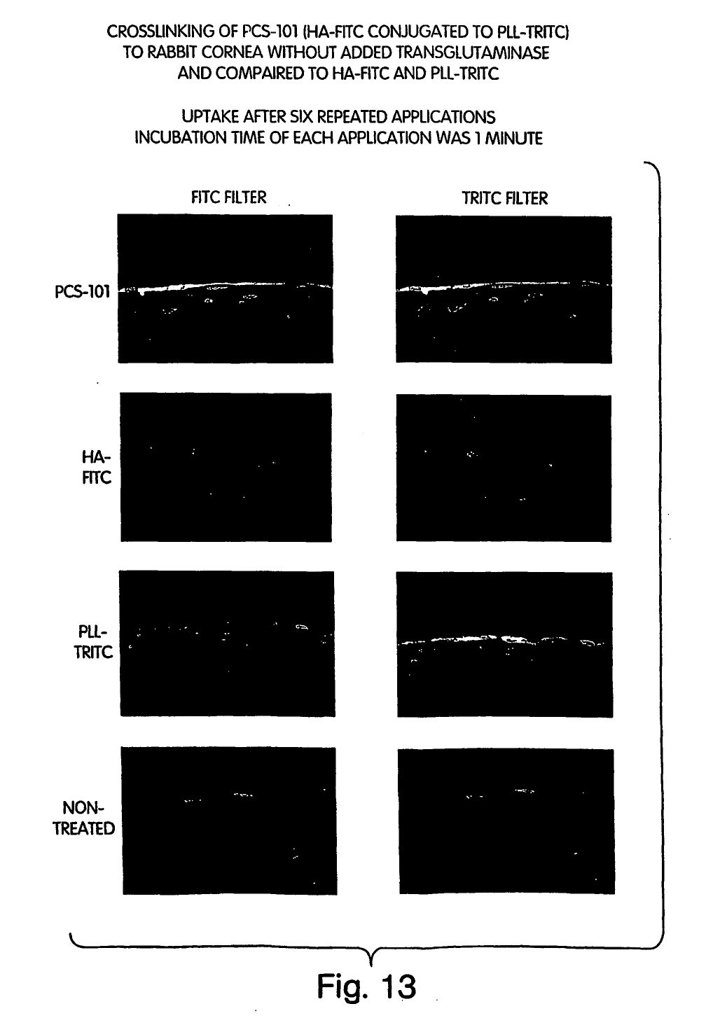

FIG. 13 demonstrates the double staining of FITC and TRITC when HA-PLL conjugates are applied to rabbit cornea in the absence of exogenous transglutaminase (first panel). The photographs were taken following six repeated applications of the conugate with an incubation time of 1 minute per application. The HA was further conjugated to FITC while the PLL was further conjugated to TRITC. No staining was observed when only HA-FITC was used (second panel). PLL-TRITC labelled the cornea (third panel). Non-treated tissues demonstrated only background fluorescence (fourth panel). This staining pattern indicates that the FITC fluorsence observed using PCS-101 was due to PLL binding to the cornea, as indicated by the third panel, and not from non-specific binding of the HA-FITC.

Conclusions:

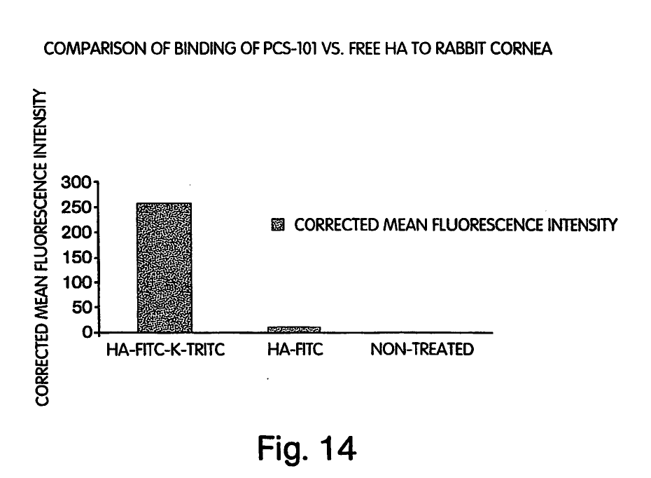

The data indicate that rosslinking of PCS-101 (HA-PLL conjugate) to the superficial layer of rabbit cornea increased with an increase in the number of applications. In contrast, free HA (HA-FITC) did not significantly bind to the superficial layer of rabbit cornea. This was evidenced by the fact that the fluorescence intensity on the tissue sections remained at background level and did not increase with increased applications. After six repeated applications, the amount of PCS-101 retained by rabbit cornea is approximately 24 times higher than that of free HA as measured by their relative corrected mean FITC fluorescence intensities (See FIG. 14).

### Equivalents

It will be understood that various modifications may be made to the embodiments disclosed herein. Therefore, the above description should not be construed as limiting, but merely as exemplifications of preferred embodiments. Those skilled in the art will envision other modifications within the scope of the claims appended hereto.

All references, patents and patent applications disclosed herein are incorporated by reference in their entirety.

APPENDIX A
REACTIVITY OF COMMERCIALLY AVAILABLE CROSS-LINKERS

| | Reactive Towards | | | | | | | | |
|---|---|---|---|---|---|---|---|---|---|
| | | | | Non- | | | | | |
| Double-Agent | | | | selective | | | | | |
| Cross-Linker | —NH₂ | —SH | | (Photo- | —COOH | Cleavable By | | | |
| Acronym | Aminos | Sulfhydryls | Carbohydrates | reactive) | Carboxyls | Thiols | Base | Periodate | Hydroxylamine |
| ABH | | | X | X | | | | | |
| ANB-NOS | X | | | X | | | | | |
| APDP | | X | | X | | X | | | |
| APG | | | | X | | | | | |
| ASIB | | X | | X | | | | | |
| ASBA | | | | X | X | | | | |
| BASED | | | | X | | X | | | |
| BS¹ | X | | | | | | | | |
| BMH | | X | | | | | | | |
| BSOCOES | X | | | | | | X | | |
| DFDNB | X | | | | | | | | |
| DMA | X | | | | | | | | |
| DMP | X | | | | | | | | |
| DMS | X | | | | | | | | |
| DPDPB | | X | | | | X | | | |
| DSG | X | | | | | | | | |
| DSP | X | | | | | X | | | |
| DSS | X | | | | | | | | |
| DST | X | | | | | | | X | |
| DTBP | X | | | | | X | | | |
| DTSSP | X | | | | | X | | | |
| EDC | X | | | | X | | | | |
| EGS | X | | | | | | | | X |
| GMBS | X | X | | | | | | | |
| HSAB | X | | | X | | | | | |
| LC-SPDP | X | X | | | | | | | |
| MBS | X | X | | | | X | | | |
| H₂C₂H | | X | X | | | | | | |
| MPBH | | X | X | | | | | | |
| NHS-ASA | X | | | X | | | | | |
| ^{γ}PDPH | | X | X | | | X | | | |
| PNP-DTP | X | | | X | | | | | |
| SADP | X | | | X | | X | | | |
| SAED | X | | | X | | X | | | |
| SAND | X | | | X | | X | | | |
| SANPAH | X | | | X | | | | | |
| SASD | X | | | X | | X | | | |
| SDBP | X | | | | | | | | |
| SIAB | X | X | | | | | | | |
| SMCC | X | X | | | | | | | |
| SHBP | X | X | | | | | | | |
| SMPT | X | X | | | | | | | |
| SPDP | X | X | | | | X | | | |
| Sulfo-BSOCOES | X | | | | | | X | | |
| Sulfo-DST | X | | | | | | | X | |
| Sulfo-EGS | X | | | | | | | | X |
| Sulfo-GMBS | X | X | | | | | | | |
| Sulfo-HSAB | X | | | X | | | | | |
| Sulfo-LC-SPDP | X | X | | | | X | | | |
| Sulfo-MBS | X | X | | | | | | | |
| Sulfo-NHS-ASA | X | | | X | | | | | |
| Sulfo-NHS-LC-ASA | X | | | X | | | | | |
| Sulfo-SADP | X | | | X | | X | | | |
| Sulfo-SAMCA | X | | | X | | | | | |
| Sulfo-SANPAH | X | | | X | | | | | |
| Sulfo-SAPB | X | | | X | | | | | |
| Sulfo-SIAB | X | X | | | | | | | |
| Sulfo-SMCC | X | X | | | | | | | |
| Sulfo-SMBP | X | X | | | | | | | |
| Sulfo-LC-SMPT | X | X | | | | | | | |

We claim:

1. A composition comprising a conjugate of hyaluronic acid and a linking molecule that is a substrate of transglutaminase, and free hyaluronic acid, wherein the free hyaluronic acid and the conjugate are present in a molar ratio of at least 2.

2-27. (canceled)

28. A pharmaceutical composition comprising hyaluronic acid covalently linked to a linking molecule that is a substrate of transglutaminase, wherein the linking molecule is uncomplexed.

29-54. (canceled)

55. A composition comprising a conjugate of hyaluronic acid and a linking molecule that is a substrate of transglutaminase, in an eye dropper bottle.

56-107. (canceled)

108. A method for treating a subject comprising administering to an eye of a subject having or at risk of having dryness of the eye an effective amount of a conjugate of hyaluronic acid and a linking molecule that is a substrate of transglutaminase.

109. The method of claim 108, wherein the conjugate is provided in a form selected from the group consisting of an eye dropper, a contact lens solution, an ophthalmic ointment, an eye pack, and a contact lens.

110. A method of treating a subject comprising administering to an oral cavity of a subject having or at risk of having dryness of the oral cavity an effective amount of a conjugate of hyaluronic acid and a linking molecule that is a substrate of transglutaminase.

111-114. (canceled)

115. The method of claim 108, wherein the effective amount is less than 0.05 μg/kg per day.

116. The method of claim 108, wherein the linking molecule has at least two contiguous aliphatic amines, at least three contiguous aliphatic amines, at least four contiguous aliphatic amines, at least five aliphatic amines, or at least six aliphatic amines.

117. The method of claim 108, wherein the linking molecule is native polylysine.

118. The method of claim 108, wherein polylysine is selected from the group consisting of poly-L-lysine, poly-D-lysine, and poly-DL-lysine.

119. The method of claim 108, wherein the linking molecule is a derivative of polylysine.

120-123. (canceled)

124. The method of claim 108, wherein the hyaluronic acid is native hyaluronic acid.

125. The method of claim 108, wherein the hyaluronic acid is a derivative of hyaluronic acid selected from the group consisting of a pharmaceutically acceptable salt of hyaluronic acid, a hyaluronic acid ester, and a sulfated hyaluronic acid.

126. The method of claim 108, wherein the hyaluronic acid has a molecular weight of at least 100,000.

127. The method of claim 108, wherein the conjugate has a negative charge to positive charge ratio of greater than 1.0.

128. The method of claim 108, wherein the conjugate is administered in a pharmaceutically acceptable carrier.

129. The method of claim 108, wherein the conjugate is administered in a pharmaceutically acceptable carrier that comprises an ophthalmic preservative.

130. The method of claim 129, wherein the ophthalmic preservative is selected from the group consisting of organic mercurials, quaternary ammonium compounds, parahydroxybenzoic acid esters, substituted alcohols and phenols.

131. The method of claim 129, wherein the organic mercurial is selected from the group consisting of phenylmercuric nitrate, phenylmercuric acetate, phenylmercuric borate, and thimerosal.

132. The method of claim 129, wherein the quaternary ammonium compound is selected from the group consisting of benzalkonium chloride, benzethonium chloride, cetyl pyridinium chloride, and polyquaternium-1 (POLYQUAD).

133. The method of claim 129, wherein the substituted alcohol and phenol is selected from the group consisting of chlorobutanol, and chlorobutanol/phenylethyl alcohol.

134. The method of claim 128, wherein the ophthalmic preservative is an antibiotic.

135. (canceled)

136. The method of claim 108, wherein the conjugate has a weight ratio selected from the group consisting of at least 90%, at least 95%, and at least 99%.

137. The method of claim 128, wherein the pharmaceutically acceptable carrier comprises arginine.

138. The method of claim 128, wherein the pharmaceutically acceptable carrier has a pH of at least 6.5.

139. The method of claim 128, wherein the pharmaceutically acceptable carrier has an osmolality of at least 280 mOsm.
